SEQUENCE LISTING (4 sequences)

SEQ ID NO: 1 - RNA, 22 nt, Homo sapiens
ucucccaacccuuguaccagug

SEQ ID NO: 2 - RNA, 22 nt, Homo sapiens
uagcagcacaucaugguuuaca

SEQ ID NO: 3 - RNA, 22 nt, Homo sapiens
uagcagcacguaaauauuggcg

SEQ ID NO: 4 - RNA, 22 nt, Homo sapiens
cuuagcagguuguauuaucauu

(19) United States
(12) Patent Application Publication
(10) Pub. No.: US 2016/0298198 A1
(43) Pub. Date: Oct. 13, 2016

(54) METHOD FOR PREDICTING DEVELOPMENT OF MELANOMA BRAIN METASTASIS

(71) Applicant: NEW YORK UNIVERSITY, New York, NY (US)

(72) Inventors: Eva Hernando-Monge, New York, NY (US); Douglas Hanniford, New York, NY (US); Hua Zhong, Short Hills, NJ (US); Iman Osman, Jersey City, NJ (US)

(73) Assignee: New York University, New York, NY (US)

(21) Appl. No.: 15/036,718

(22) PCT Filed: Nov. 13, 2014
(86) PCT No.: PCT/US14/65360

§ 371 Date: May 13, 2016

Related U.S. Application Data
(60) Provisional application No. 61/905,004, filed on Nov. 15, 2013.

(51) Int. Cl. C12Q 1/68; A61K 31/506; A61N 5/10; C12N 15/113; A61K 31/7105; A61K 31/437; C07K 16/28

(52) U.S. Cl. CPC C12Q 1/6886; A61K 31/437; A61K 31/506; C07K 16/2818; C07K 16/2827; C12N 15/113; A61K 31/7105; A61N 5/10; C12N 2310/141; C12N 2320/30; C12N 2310/113; C12Q 2600/178; C12Q 2600/158; C12Q 2600/118

(57) ABSTRACT

The invention relates to miRNA-based methods for prognosis of melanoma brain metastasis and related methods and kits. In one embodiment, the invention provides a method for predicting the likelihood of developing melanoma brain metastasis (B-Met) in a subject diagnosed with primary melanoma, said method comprising determining the levels of miR-150-5p, miR-15b-5p, miR-16-5p, and miR-374b-3p in a primary melanoma sample collected from the subject.

### CROSS-REFERENCE TO RELATED APPLICATIONS

This application claims priority to U.S. Provisional Application Ser. No. 61/905,004, filed Nov. 15, 2013, which is herein incorporated by reference in its entirety.

### STATEMENT AS TO FEDERALLY SPONSORED RESEARCH

This invention was funded, in part, by Department of Defense (DOD) Award W81XWH-10-1-0803, and National Institutes of Health (NIH) and National Cancer Institute Award (NCI) Award 1R01CA163891-01A1. Accordingly, the U.S. government has certain rights to this invention.

### SEQUENCE LISTING

The instant application contains a Sequence Listing which has been submitted electronically in ASCII format and is hereby incorporated by reference in its entirety. Said ASCII copy, created on Nov. 12, 2014, is named 243735-000132_SL.txt and is 840 bytes in size.

### FIELD OF THE INVENTION

The invention relates to miRNA-based methods for prognosis of melanoma brain metastasis and related methods and kits.

### BACKGROUND OF THE INVENTION

Melanoma is a prevalent cancer of increasing incidence globally (Siegel et al., CA: a cancer journal for clinicians 2013, 63:11-30) with a cost of nearly a billion dollars annually to the health care system. (Guy et al., American Journal of Preventive Medicine 2012, 43:537-45). While the majority of patients are diagnosed with localized melanoma and effectively cured through surgical management, melanoma has a propensity to spread, with even some of the smallest, thinnest tumors metastasizing. (Balch et al., Journal of Clinical Oncology 2009, 27:6199-405). In particular, brain metastasis (B-Met) is a major clinical burden of metastatic melanoma patients. B-Met occurs in 5-15% of all melanoma patients and roughly 50% of metastatic melanoma patients, and is the cause of approximately 50% of melanoma deaths. (Flanigan et al., Clinics in Dermatology 2013, 31:264-81). The median age of melanoma B-Met diagnosis is 57, and B-Met is rapidly fatal, with median survival of 4 months after diagnosis, despite current treatment strategies. (Fife et al., J Clin Oncol 2004, 22:1293-300). Approaches to better stratify patients by their risk of B-Met development may yield substantial benefit of life years gained through intensified surveillance and aggressive treatment of the highest risk patients. However, currently used histopathological parameters are insufficient predictors of which primary melanoma patients will develop advanced disease, particularly B-Met. Consequently, identification of robust prognostic markers of melanoma B-Met represents an important clinical challenge in melanoma biology.

Recent studies have explored the potential of microRNA (miRNA), small, non-coding RNA which regulate translation and stability of protein-coding messenger RNA, as biomarkers of disease outcome (Iorio & Croce, EMBO Mol Med 2012, 4:143-59). Their diagnostic or prognostic value resides in their lineage/tumor type specificity, stability in formalin-fixed, paraffin embedded (FFPE) tissues and body fluids, and ease of isolation and quantification (Xi et al., RNA 2007, 13; Klopfleisch et al., Histology and Histopathology 2011, j26:797-1607). Rather than mere passenger alterations, burgeoning literature has revealed fundamental roles for some miRNA in the tumorigenicity of diverse cancers (Croce C M, Calin G A. Cell 2005, 122:6-7; Segura et al., Carcinogenesis 2012, Pencheva et al., Cell 2012, 151:1068-82; Gaziel-Sovran et al., Cancer Cell 2011, 20:104-22).

### SUMMARY OF THE INVENTION

As follows from the Background section, above, there is an unmet need in the art for compositions and methods for predicting melanoma brain metastasis at the time of primary tumor diagnosis.

The present invention addresses these and other needs by providing the methods and kits described below.

In one embodiment, the invention provides a method for predicting the likelihood of developing melanoma brain metastasis (B-Met) in a subject diagnosed with primary melanoma, said method comprising:

a. determining the levels of miR-150-5p, miR-15b-5p, miR-16-5p, and miR-374b-3p in a primary melanoma sample collected from the subject;

b. calculating a B-Met prognostic score based on a weighted summation of the miRNA levels determined in step (a) and melanoma stage of the primary melanoma sample collected from the subject;

c. comparing the B-Met prognostic score calculated in step (b) with a corresponding control value;

d. (i) identifying the subject as being at high risk of developing B-Met if the B-Met prognostic score calculated in step (b) is higher than the corresponding control value or (ii) identifying the subject as being at low risk of developing B-Met if the B-Met prognostic score calculated in step (b) is same or lower than the corresponding control value.

Non-limiting examples of the methods for determining the levels of the miRNA in the above method of the invention include, e.g., hybridization, array-based assays, RT-PCR-based assays, and sequencing.

In a further embodiment, the invention provides a method for predicting the likelihood of developing melanoma brain metastasis (B-Met) in a subject diagnosed with primary melanoma, said method comprising:

a. determining the levels of miR-150-5p, miR-15b-5p, miR-16-5p, and miR-374b-3p in a primary melanoma sample collected from the subject by an RT-PCR-based assay;

b. calculating a B-Met prognostic score based on a weighted summation of the miRNA levels determined in step (a) and melanoma stage of the primary melanoma sample collected from the subject using the formula

Score=−0.51 miR-150+0.65 miR15b−0.96miR-16−0.24 miR-374+0.47*stage;

c. comparing the B-Met prognostic score calculated in step (b) with a corresponding control value;

d. (i) identifying the subject as being at high risk of developing B-Met if the B-Met prognostic score calculated in step (b) is higher than the corresponding control value or (ii) identifying the subject as being at low risk of developing B-Met if the B-Met prognostic score calculated in step (b) is same or lower than the corresponding control value.

In another embodiment, the invention provides a method for predicting the likelihood of developing melanoma brain metastasis (B-Met) in a subject diagnosed with primary melanoma, said method comprising:

a. determining the levels of miR-150-5p, miR-15b-5p, miR-16-5p, and miR-374b-3p in a primary melanoma sample collected from the subject by a microarray-based assay;

b. calculating a B-Met prognostic score based on a weighted summation of the miRNA levels determined in step (a) and melanoma stage of the primary melanoma sample collected from the subject using the formula

Score=−0.51 miR-150+0.65 miR15b−0.96 miR-16−0.24 miR-374+0.47*stage;

c. comparing the B-Met prognostic score calculated in step (b) with a corresponding control value;

d. (i) identifying the subject as being at high risk of developing B-Met if the B-Met prognostic score calculated in step (b) is higher than the corresponding control value or (ii) identifying the subject as being at low risk of developing B-Met if the B-Met prognostic score calculated in step (b) is same or lower than the corresponding control value.

In one embodiment of any of the above methods, if there is more than one sample in the measured cohort, all miRNAs are standardized into mean 0 and unit variance variables by subtracting the mean and dividing by the standard deviation (SD) of the cohort, before fitting into the weighted summation formula.

In one specific embodiment of any of the above methods, the subject has been diagnosed with primary cutaneous melanoma. In one specific embodiment of any of the above methods, the subject has been diagnosed with stage I, stage II or stage III primary melanoma.

In one specific embodiment of any of the above methods, the melanoma stage is determined according to the American Joint Committee on Cancer (AJCC) 2009 final guidelines. In one specific embodiment of any of the above methods, the method further comprises determining melanoma stage of the primary melanoma sample collected from the subject prior to step (b) (e.g., according to the American Joint Committee on Cancer (AJCC) 2009 final guidelines).

In one specific embodiment of any of the above methods, the control value is a predetermined standard. In another specific embodiment of any of the above methods, the control value is the mean of prognostic probabilities calculated for the same miRNA in several similarly prepared melanoma samples isolated from subjects with low risk of developing B-Met.

In one specific embodiment of any of the above methods, the method further comprises normalizing the levels of miRNA determined in step (a) to one or more normalizer RNA. The normalizer RNA can be, for example, miRNA. In one specific embodiment, the normalizer miRNA is the combination of let-7e-5p, miR-30d-5p, and miR-423-3p. In one specific embodiment involving the normalizer RNA, the method further comprises:

(i) calculating the mean expression Ct/Cp for the one or more normalizer miRNA to generate a normalizer Ct/Cp;
(ii) subtracting test miRNA Ct/Cp from the normalizer Ct/Cp calculated in step (i) to generate normalized delta Ct/Cp, and
(iii) using the normalized delta Ct/Cp calculated in step (ii) in step (b).

In one specific embodiment of any of the above methods, the method further comprises recruiting the subject in a clinical trial.

In one specific embodiment of any of the above methods, the method further comprises conducting a regular surveillance of the subject determined in step (e) as being at high risk of developing B-Met for the presence of B-Met. In one specific embodiment of any of the above methods, the method further comprises increasing the frequency of surveillance of the subject determined in step (e) as being at high risk of developing B-Met, as compared to the subject determined as being at low risk of developing B-Met. Such surveillance can include, for example, brain imaging and/or clinical evaluation.

In one specific embodiment of any of the above methods, the method further comprises conducting an extensive primary tumor staging of the subject determined in step (e) as being at high risk of developing B-Met.

In one specific embodiment of any of the above methods, the method further comprises administering to the subject determined in step (e) as being at high risk of developing B-Met a treatment specifically targeted at prevention or treatment of B-Met. Non-limiting examples of useful treatments include, for example, cranial irradiation (can be done prophylactically), Interferon alpha, BRAF inhibitors (e.g., vemurafenib or dabrafenib), MEK inhibitors (e.g., trametinib), imatinib, nilotinib, dacarbazine, temozolomide, and immunotherapy agents (e.g., anti-CTLA4, anti-PD1, and anti-PD-L1).

In one specific embodiment of any of the above methods, the method further comprises a step of collecting the primary melanoma sample from the subject prior to step (a).

In one specific embodiment of any of the above methods, prior to determining miRNA level, the miRNA is purified from the melanoma sample.

In one specific embodiment of any of the above methods, the method further comprises the step of reducing or eliminating degradation of the miRNA.

In a separate embodiment, the invention provides a method for prevention or treatment of melanoma brain metastasis (B-Met) in a subject in need thereof comprising increasing the level and/or activity of one or more miRNA selected from the group consisting of miR-150-5p, miR-16-5p, and miR-374b-3p in the melanoma cells of the subject. In one specific embodiment of this method, increasing the level and/or activity of said one or more miRNA is achieved by a method selected from the group consisting of over-expressing miRNA or mature miRNA mimic, sense-based oligonucleotides, and modified-oligonucleotide mimics.

In another embodiment, the invention provides a method for prevention or treatment of melanoma brain metastasis (B-Met) in a subject in need thereof comprising decreasing the level and/or activity of miR-15b-5p in the melanoma cells of the subject. In one specific embodiment of this method, decreasing the level and/or activity of said miRNA is achieved using an RNAi molecule or an antisense oligonucleotide.

In one embodiment of the above two prevention/treatment methods, the method further comprises administering to the subject an additional treatment specifically targeted at prevention or treatment of B-Met. Non-limiting examples of useful additional treatments include, for example, cranial irradiation (can be done prophylactically), Interferon alpha, BRAF inhibitors (e.g., vemurafenib or dabrafenib), MEK inhibitors (e.g., trametinib), imatinib, nilotinib, dacarbazine, temozolomide, and immunotherapy agents (e.g., anti-CTLA4, anti-PD1, and anti-PD-L1).

In a separate embodiment, the invention provides a method for quantifying tumor-infiltrating lymphocytes (TILs) in a tumor sample collected from a subject, wherein the tumor expresses low levels or does not express miR-150-5p, said method comprising determining the level of miR-150-5p in the tumor sample.

In another embodiment, the invention provides a method for quantifying lymphocyte response to a tumor in a subject, wherein the tumor expresses low levels or does not express miR-150-5p, said method comprising determining the level of miR-150-5p in a tumor sample collected from the subject.

Non-limiting examples of tumors expressing low levels or no miR-150-5p for which the above two lymphocyte quantifying methods can be used include, e.g., melanoma, prostate cancer, breast cancer, hepatocellular carcinoma, renal cell carcinoma, and colorectal carcinoma.

In one specific embodiment of the above two lymphocyte quantifying methods, the level of miR-150-5p in the tumor sample is normalized to the level of miR-150-5p in the surrounding stroma. In one specific embodiment of the above two lymphocyte quantifying methods, the level of miR-150-5p is determined using a method selected from the group consisting of hybridization, array-based assays, RT-PCR-based assays, and sequencing. In one specific embodiment of the above two lymphocyte quantifying methods, prior to determining the level of miR-150-5p, miRNA is purified from the tumor sample. In one specific embodiment of the above two lymphocyte quantifying methods, the method further comprises the step of reducing or eliminating degradation of miRNA in the tumor sample.

In one specific embodiment of any of the methods of the present invention, the subject is human. In another specific embodiment of any of the methods of the present invention, the subject is an experimental animal.

In a separate embodiment, the invention provides a kit comprising primers and/or probes specific for miR-150-5p, miR-15b-5p, miR-16-5p, and miR-374b-3p.

In one specific embodiment, any of the above two kits further comprise miRNA isolation or purification means. In one specific embodiment, any of the above two kits further comprise instructions for use.

### BRIEF DESCRIPTION OF THE DRAWINGS

FIGS. 1A-I illustrate that a 4-miRNA signature accurately stratifies patients by brain metastasis-free and overall survival. Risk scores for individual patients were defined as the linear combination of model predictors weighted by regression coefficients. An optimal risk score cutoff selected by using the Youden Index of the discovery cohort ROC curve was chosen to classify patients into high- and low-risk groups. Group data were plotted in Kaplan-Meier survival curves and statistical analysis performed by Wilcoxon tests for brain metastasis-free and overall survival of training (A), validation (B), and independent (C) patient cohorts. Panels D-I show that the 4-miRNA signature can be utilized to stratify patients within individual disease stages as to B-Met-free survival and overall survival; analyses for stage I are shown in (D) and (G), stage II in (E) and (H), and stage III in (F) and (I).

FIGS. 2A-D show that miR-150-5p expression correlates with CD45⁺ TILs in primary melanoma tissues. miR-150-5p levels were measured by RT-qPCR (Applied Biosystems, now part of Life Technologies, Catalog #4427975 part ID 000473) in a subset of 82 samples (23 B-Met, 20 extracranial metastases, and 39 non-recurrences). Differences in miR-150-5p expression were evaluated by two-tailed unpaired T-test for samples with ‘non-brisk’ (n=27) vs. ‘brisk’ (n=55) TIL responses (p=0.006) (A) and B-Met (n=23) vs. Non-B-Met (n=59) (p=0.025) (B). A subset of 48 primary melanomas (B-Met n=12, Non-B-Met=36) previously analyzed for miR-150-5p expression and TILs were stained for the leukocyte marker, CD45 (Leukocyte Common Antigen, LCA), clone RP2/18 (Cat. 760-2505, Ventana Medical Systems Tucson, Ariz. USA). miR-150-5p levels were plotted against the absolute (C) and relative (D) number of intratumoral CD45⁺ cells (p=0.007 and p=0.016 respectively).

FIGS. 3A-C provide summaries of data supporting various embodiments of the present invention. The clinical characteristics of patients used in this study are provided in (A). The members hazard ratios, and p values of the 4-miRNA signature predicting development of melanoma brain metastasis are provided in (B). The 4-miRNA signature in combination with stage improves prognosis of brain metastasis-free survival as shown in (C).

FIGS. 4A-D illustrate plotted representative images of hematoxylin & eosin (H&E) (A, B) or CD45 (C, D) stained sections of primary melanomas that metastasized to brain, shown with non-brisk TILs (A, C) or have not yet metastasized to brain, shown with brisk TILs (B, D). Most of the signal evident in the B-Met/non-brisk TIL CD45 image is pigmentation.

FIG. 5 shows median risk scores supporting that the 4-miRNA classifier is partially reflective of brain tropism. Patients from all cohorts were divided into four groups (non-recurrent (n=115), non-brain recurrent (n=67), concomitant brain metastatic (n=51), and isolated, first-site brain metastatic (n=16)) and risk scores plotted in box & whiskers format. Whiskers are min-max, excluding outliers (defined as above 1.5 IQR away from the 25% and 75% quantiles). Statistical analysis was performed by Kruskal-Wallis rank-sum test.

FIG. 6 illustrates that the 4-miRNA classifier converts well between miRNA quantification platforms. Patient prediction scores were obtained through application of regression coefficients of the Cox Proportional Hazards logistic regression model on the standardized miRNA expressions. (A) To assess level of concordance, prediction score correlations for the same set of patients measured by microarray and RT-qPCR platforms are plotted and correlation measured (coefficient is estimated at 0.412 (p<0.001)). Statistical analysis was performed by Spearman correlation.

FIGS. 7A-K provide summaries of data supporting various embodiments of the present invention. Clinical characteristics (cohort, gender, age, primary tumor thickness, mitotic index, ulceration status, histological subtype, anatomical site, stage at DX) of individual patients 1-50 (A), 51-100 (B), 101-150 (C), 151-200 (D), 201-249 (E); and clinical characteristics (recurrence, time to 1st recurrence, site of 1st recurrence, B-Met, time to B-Met, site of first recurrence in brain, last status, F/U time, cause of death) of individual patients 1-50 (F), 51-100 (G), 101-150 (H), 151-200 (I), 201-249 (J). (&: Mitoses per mm²⁻ Few=1-2; Moderate=3-4; Many=5+); (#: NM=Nodular melanoma; SSM=Superficial Spreading Melanoma; AL=Acral Lentiginous Melanoma; DM=Desmoplastic Melanoma; 0=Other; U=Unknown); (A: LR=local recurrence; RS=regional skin; DS=distant skin; RLN=regional lymph node; DLN=distant lymph node; DV=distant visceral). Coefficients of the 4-miRNA signature predicting brain metastasis using stage as a categorical variable (K).

### DETAILED DESCRIPTION OF THE INVENTION

The present invention is based on the unexpected discovery that a molecular signature of expression of 4 microRNA (miRNA) detected in primary melanoma tissue at the time of the initial diagnosis, in combination with stage, can robustly stratify patients by their risk of developing melanoma brain metastasis (B-Met). As discussed in more detail in the Examples section, below, the prognostic value of miRNA expression was analyzed in primary melanoma, specifically for its ability to distinguish patients who develop melanoma B-Met from those with non-recurrent tumors and tumors metastatic to other sites. The current invention provides a 4-miRNA signature (miR-150-5p, miR-15b-5p, miR-16-5p, and miR-374b-3p) that, in combination with stage, distinguishes primary tumors that metastasize to the brain from non-recurrent and non-brain-metastatic primary tumors (AUC=84.3% of the ROC curve).

Application of this model to discriminate brain metastasis in validation and independent cohorts yields comparable results. Corresponding Kaplan-Meier curves of high-vs. low-risk patients displays a clear separation in brain-metastasis-free survival (training: p<0.001, validation: p<0.001, independent: p=0.002).

The core B-Met prognostic signature of the present invention contains the following 4 mature miRNA:

| | Corresponding | Corresponding sequence | SEQ |
|---|---|---|---|
| | name for | for mature human miRNA | ID |
| miRNA | human miRNA | (miRBase Accession No.) | NO |
| miR- | hsa-miR-150- | UCUCCCAACCCUUGUACCAGUG | 1 |
| 150-5p | 5p | (MIMAT0000451) | |
| miR- | hsa-miR-15b- | UAGCAGCACAUCAUGGUUUACA | 2 |
| 15b-5p | 5p | (MIMAT0000417) | |
| miR- | hsa-miR-16- | UAGCAGCACGUAAAUAUUGGCG | 3 |
| 16-5p | 5p | (MIMAT0000069) | |
| miR- | hsa-miR-374b- | CUUAGCAGGUUGUAUUAUCAUU | 4 |
| 374b-3p | 3p | (MIMAT0004956) | |

The present invention also provides that the expression of a key lymphocyte miRNA, miR-150-5p, which is less abundant in primary melanomas metastatic to brain, correlates with the presence of CD45⁺ tumor infiltrating lymphocytes.

### DEFINITIONS

As used herein, the terms “melanoma brain metastasis” and “B-Met)” refer to a spread of melanoma cancer cells beyond the site of the primary tumor into any region of the brain.

The terms “microRNA” or “miRNA” as used herein refer to a class of small approximately 22 nt long non-coding RNA molecules. They play important roles in the regulation of target genes by binding to complementary regions of messenger transcripts (mRNA) to repress their translation or regulate degradation (Griffiths-Jones Nucleic Acids Research, 2006, 34, Database issue: D140-D144). Frequently, one miRNA can target multiple mRNAs and one mRNA can be regulated by multiple miRNAs targeting different regions of the 3′ UTR. Once bound to an mRNA, miRNA can modulate gene expression and protein production by affecting, e.g., mRNA translation and stability (Baek et al., Nature 455(7209):64 (2008); Selbach et al., Nature 455(7209):58 (2008); Ambros, 2004, Nature, 431, 350-355; Bartel, 2004, Cell, 116, 281-297; Cullen, 2004, Virus Research., 102, 3-9; He et al., 2004, Nat. Rev. Genet., 5, 522-531; and Ying et al., 2004, Gene, 342, 25-28). Information on most currently known miRNAs can be found in the miRNA database miRBase (available at the World Wide Web at mirbase.org).

The term “miRNA array” refers to a multiplex technology used in molecular biology and in medicine. It consists of an arrayed series of multiple (e.g., up to 2000) microscopic spots of oligonucleotides, each containing a specific sequence (probe) complementary to a particular target miRNA. After probe-target hybridization under high-stringency conditions the resulting hybrids are usually detected and quantified by quantifying fluorophore-, silver-, or chemiluminescence-labeled targets to determine relative abundance of miRNA. In the methods of the present invention, both custom-made and commercially available miRNA arrays can be used. Non-limiting examples of useful commercially available miRNA arrays (based on various methods of target labeling, hybrid detection and analysis) include arrays produced by Exiqon, Affymetrix, Agilent, Illumina, Invitrogen, Febit, and LC Sciences.

The term “next generation sequencing technologies” broadly refers to sequencing methods which generate multiple sequencing reactions in parallel. This allows vastly increased throughput and yield of data. Non-limiting examples of commonly used next generation sequencing platforms include Helicos small RNA sequencing, miRNA BeadArray (Illumina), Roche 454 (FLX-Titanium), and ABI SOLiD.

The term “combined levels of the miRNAs” as used herein refers to the linear combinations of miRNAs levels. The linear combinations of miRNAs levels can be calculated using any method known in the art. For example, as specified in the Examples section, below, the coefficients required for such linear combinations can be calculated using the logistic regression method, i.e., β1*x1+β2*x2+ . . . +βk*xk, where β's are the coefficients from logistic regression model, and x's are the levels of miRNAs (see, e.g., Steyerberg (2009) Clinical Prediction Models, Springer, N.Y.). The coefficients can be positive or negative. The “combined levels”, or the score, is always positively associated with the risk of recurrence. Thus, patients with higher score have a higher probability of recurrence. The logistic regression method can also include more clinical predictors if necessary. The performance of logistic regression models is measured using the area under the Receiving Operative Characteristic (ROC) curves: the larger the area under the ROC curve, the better performance of the model. The best performed model would yield the coefficients required to calculate the linear combination of levels of candidate miRNAs.

In the context of the present invention insofar as it relates to melanoma and melanoma metastasis, the terms “treat”, “treatment”, and the like mean to relieve or alleviate at least one symptom associated with such condition, or to slow or reverse the progression of melanoma or melanoma brain metastasis, or to arrest, prevent or delay the onset (i.e., the period prior to clinical manifestation) and/or reduce the risk of developing or worsening of melanoma or melanoma brain metastasis.

An “individual” or “subject” or “animal”, as used herein, refers to humans, veterinary animals (e.g., cats, dogs, cows, horses, sheep, pigs, etc.) and experimental animal models of melanoma. In a preferred embodiment, the subject is a human.

The term “purified” as used herein refers to material that has been isolated under conditions that reduce or eliminate the presence of unrelated materials, i.e., contaminants, including native materials from which the material is obtained. For example, RNA purification includes elimination of proteins, lipids, salts and other unrelated compounds. Besides, for some methods of analysis a purified miRNA is preferably substantially free of other RNA oligonucleotides contained in tumor samples (e.g., rRNA and mRNA fragments, etc.). As used herein, the term “substantially free” is used operationally, in the context of analytical testing of the material. Preferably, purified material substantially free of contaminants is at least 50% pure; more preferably, at least 90% pure, and still more preferably at least 99% pure. Purity can be evaluated by chromatography, gel electrophoresis, composition analysis, biological assay, and other methods known in the art.

As used herein, the term “similarly processed” refers to samples (e.g., tumor samples or purified miRNAs) which have been obtained using the same protocol.

The term “associated with” is used to encompass any correlation, co-occurrence and any cause-and-effect relationship.

The term “about” or “approximately” means within a statistically meaningful range of a value. Such a range can be within an order of magnitude, preferably within 50%, more preferably within 20%, still more preferably within 10%, and even more preferably within 5% of a given value or range. The allowable variation encompassed by the term “about” or “approximately” depends on the particular system under study, and can be readily appreciated by one of ordinary skill in the art.

In accordance with the present invention there may be employed conventional molecular biology, microbiology, and recombinant DNA techniques within the skill of the art. Such techniques are explained fully in the literature (e.g., Sambrook, Fritsch & Maniatis, Molecular Cloning: A Laboratory Manual, Second Edition. Cold Spring Harbor, N.Y.: Cold Spring Harbor Laboratory Press, 1989 (herein “Sambrook et al., 1989”); DNA Cloning: A Practical Approach, Volumes I and II (D. N. Glover ed. 1985); Oligonucleotide Synthesis (M. J. Gait ed. 1984); Nucleic Acid Hybridization [B. D. Hames & S. J. Higgins eds. (1985)]; Transcription And Translation [B. D. Hames & S. J. Higgins, eds. (1984)]; Animal Cell Culture [R. I. Freshney, ed. (1986)]; Immobilized Cells And Enzymes [IRL Press, (1986)]; B. Perbal, A Practical Guide To Molecular Cloning (1984); Ausubel, F. M. et al. (eds.); Current Protocols in Molecular Biology. John Wiley & Sons, Inc., 1994; Kunkel, Proc. Natl. Acad. Sci. USA 82: 488-492 (1985); U.S. Pat. No. 5,071,743; Fukuoka et al., Biochem. Biophys. Res. Commun. 263: 357-360 (1999); Kim and Maas, BioTech. 28: 196-198 (2000); Parikh and Guengerich, BioTech. 24: 428-431 (1998); Ray and Nickoloff, BioTech. 13: 342-346 (1992); Wang et al., BioTech. 19: 556-559 (1995); Wang and Malcolm, BioTech. 26: 680-682 (1999); Xu and Gong, BioTech. 26: 639-641 (1999); U.S. Pat. No. 5,789,166; U.S. Pat. No. 5,932,419; Hogrefe, Strategies 14. 3: 74-75 (2001); U.S. Pat. No. 5,702,931; U.S. Pat. No. 5,780,270; U.S. Pat. No. 6,242,222; Angag and Schutz, Biotech. 30: 486-488 (2001); Wang and Wilkinson, Biotech. 29: 976-978 (2000); Kang et al., Biotech. 20: 44-46 (1996); Ogel and McPherson, Protein Engineer. 5: 467-468 (1992); Kirsch and Joly, Nucl. Acids. Res. 26: 1848-1850 (1998); Rhem and Hancock, J. Bacteriol. 178: 3346-3349 (1996); Boles and Miogsa, Curr. Genet. 28: 197-198 (1995); Barrenttino et al., Nuc. Acids. Res. 22: 541-542 (1993); Tessier and Thomas, Meths. Molec. Biol., 1996, 57: 229-237; Pons et al., Meth. Molec. Biol., 1997, 67: 209-218).

### Prognostic Methods of the Invention

The present invention provides novel highly sensitive methods for predicting the likelihood of developing melanoma brain metastasis (B-Met) at the time of diagnosis in a subject.

### Parameter Calculation (RT-qPCR)

a. miRNA are amplified on a per sample basis. A threshold is set and Ct/Cp values are considered the point at which the amplification curve crosses this threshold. These are relative expression values. Expression values are normalized to control miRNA. In some embodiments, the combination of three miRNA is used as a normalizer (i.e., hsa-let-7e-5p, hsa-miR-30d-5p, and hsa-miR-423-3p). For normalizers, the mean expression Ct/Cp is calculated per sample to generate a normalizer Ct/Cp and test miRNA Ct/Cp is then subtracted from this normalizer Ct/Cp on a per sample basis to generate normalized delta Ct/Cp. These delta Ct/Cp are essentially log 2 normalized expression values. Delta Ct/Cp=normalizer−Ct/Cp.

b. To improve inter-run reproducibility of expression values:
i. Use a defined threshold for calculating Ct/Cp values
ii. Generate standard curves of each miRNA using artificial miRNA mimics or similar (e.g., Dharmacon miRIdian mimics, Life Technologies mirVana mimics, Sigma MISSION miRNA mimics) and use this standard curve during each sample analysis. This would allow for measurement of miRNA expression in absolute terms.
iii. Use of calibrator sample(s)
iv. Spike-in controls (artificial short RNA/miRNA that are not present in human samples [e.g., Exiqon UniSP2 cat#203950, UniSP4 cat#203953, UniSP5 cat#203951, cel-miR-39-3p cat#203592])—can be used at the RT step to control for RT efficiency and/or as inter-run calibrators.
   c. Analysis of expression values for risk score generation/algorithm.
   Calculation of B-Met Prognostic Score from the miRNA RT-PCR Measurements
   1. First standardize each miRNA expression by subtracting the cohort mean (column “mean” in Table 1) and dividing by cohort standard deviation (column “SD” in Table 1). The rational of this step is to bring all miRNAs on the same numerical scales. The means and SDs were estimated from 150 primary melanoma samples.

Standardized miRNA (stan−miR)=(miRNA−mean)/SD

2. The prognostic score is a weighted summation of the miRNAs, with weights listed in column “Weights of Standardized miRNAs” in Table 1. Stage is coded as 1, 2, 3 for stage I, II and III patients respectively.

Score=−3.08−0.45 stan−miR-150+1.37 stan−miR15b−0.67 stan−miR-16−0.42 stan−miR-342−0.03 stan−miR-374+1.19*stage

3. To calculate the score directly from miRNAs from RT-PCR measurements without standardization, skip step 1, and use weights listed in column “Weights of Unstandardized miRNAs” in Table 1, then move to step 3.

Score=−0.51 miR-150+0.65 miR15b−0.96 miR-16−0.24 miR-374+0.47*stage

TABLE 1
Statistics for RT-PCR miRNA

| | | | Weights | | | | Weights of |
|---|---|---|---|---|---|---|---|
| | | | of stand- | | | | Unstand- |
| | | | ardized | Std. | z | | ardized |
| miRNA | mean | SD | miRNAs | Error | value | Pr(>\|z\|) | miRNAs |
| Intercept | NA | NA | −3.08 | 0.76 | −4.07 | 0.00 | −1.15 |
| hsa.miR.150.5p | 0.53 | 1.98 | −0.45 | 0.36 | −1.27 | 0.21 | −0.23 |
| hsa.miR.15b.5p | 0.02 | 1.08 | 1.37 | 0.33 | 4.19 | 0.00 | 1.27 |
| hsa.miR.16.5p | 3.21 | 0.94 | −0.67 | 0.26 | −2.53 | 0.01 | −0.71 |
| hsa.miR.342.3p | −0.37 | 1.35 | −0.42 | 0.40 | −1.06 | 0.29 | −0.31 |
| hsa.miR.374b.3p | −9.84 | 0.97 | −0.03 | 0.21 | −0.15 | 0.88 | −0.03 |
| stage as a | NA | NA | 1.19 | 0.40 | 3.01 | 0.00 | 1.19 |
| continuous | | | | | | | |
| variable | | | | | | | |

Calculation of B-Met prognostic score from the miRNA microarray measurements 1. First, standardize each miRNA expression by subtracting the cohort mean (column “mean” in Table 2) and dividing by cohort standard deviation (column “SD” in Table 2). The rational of this step is to bring all miRNA on the same numerical scales to avoid any miRNA getting weighted higher simply because it has higher means or smaller spread. The means and SDs are estimated from a cohort of 92 primary melanomas. Therefore,

Standardized miRNA (stan−miR)=(miRNA−mean)/SD

2. The prognostic score is a weighted summation of the miRNA, with weights listed in column “Weights of Standardized miRNAs” in Table 2. Stage is coded as 1, 2, 3 for stage I, II and III patients respectively.

Score=−3.97−1.14 stan−miR-150+1.50 stan−miR15b−2.07 stan−miR-16−0.84 stan−miR-374+1.31*stage

3. To calculate the score directly from miRNA from microarray measurements without standardization, skip step 1, and use weights listed in column “Weights of Unstandardized miRNAs” in Table 2, then move to step 3.

Score=−0.51 miR-150+0.65 miR15b−0.96 miR-16−0.24 miR-374+0.47*stage

TABLE 2
Statistics for microarray miRNAs

| | | | Weights | | | | Weights of | |
|---|---|---|---|---|---|---|---|---|
| | | | of Stand- | | | | Unstand- | |
| | | | ardized | Std. | z | | ardized | Std. |
| miRNA | mean | SD | miRNA | Error | value | Pr(>\|z\|) | miRNA | Error |
| Intercept | NA | NA | −3.97 | 1.15 | −3.44 | 0.00 | −4.10 | 1.27 |
| hsa.miR.150.5p | 0.02 | 0.44 | −1.14 | 0.49 | −2.32 | 0.02 | −2.57 | 1.11 |
| hsa.miR.15b.5p | −0.06 | 0.59 | 1.50 | 0.73 | 2.05 | 0.04 | 2.54 | 1.24 |
| hsa.miR.16.5p | −0.29 | 0.98 | −2.07 | 0.90 | −2.29 | 0.02 | −2.11 | 0.92 |
| hsa.miR.342.3p | −0.23 | 0.56 | −0.69 | 0.40 | −1.73 | 0.08 | −1.24 | 0.72 |
| hsa.miR.374b.3p | 0.16 | 0.24 | −0.84 | 0.41 | −2.06 | 0.04 | −3.49 | 1.69 |
| stage as a | NA | NA | 1.31 | 0.50 | 2.61 | 0.01 | 1.31 | 0.50 |
| continuous | | | | | | | | |
| variable | | | | | | | | |

Weight of miRNA
a. miRNA are given equal weight in the model. However, individual miRNA have different weight contributions to the prognostic value of the miRNA signature as defined in Table 1 and 2.
b. The relative contributions of each miRNA is quantified by its weights and p-values in the composite score. The p-values are essentially measurements of whether the weights are statistically different from 0. From microarray-based quantifications, all four miRNA have significant p-values, thus cannot be removed from the signature without compromising the signature's performance. From the RT-PCR quantifications, miR-374b-3p has low expression in a substantial number of samples, thus resulting in low weights. It can be therefore potentially removed from the signature based on RT-PCT assay if it is not detected in a large number of samples.

### Controls and Calculation—Two Types.

a. For brain metastasis outcome, controls are patients that have not developed brain metastasis (at least within a certain time frame). In clinical use, a control can be a predetermined value (e.g., data generated during assay development). Minimization of inter-run variability (see above) is a key step to identify a well-defined risk score. In various embodiments of the methods of the invention, the risk score for developing B-Met can either represent a set of ranges or individual values.

The assay can be further developed to generate the necessary thresholds/cut-points for risk scoring. After selecting the methodology of choice for miRNA quantification (e.g., qRT-PCR), titrations of possible controls can be performed to begin to standardize the technical aspects of the assay. For example, a threshold level (from which to derive Ct/Cp values) can be standardized. Based on this, inter-run replication of titration curves can be assessed. Once established, the analysis can be expanded to a large retrospective cohort of patient samples. Preferably, a parallel prospective study should be also undertaken.

b. Control miRNA measured for data normalization purposes.

### Inclusion of Staging

a. As discussed above, the prognostic methods of the invention require combination with melanoma stage. All staging is performed for the same primary melanoma sample from which miRNA expression is measured. Staging can be performed, e.g., according to the American Joint Committee on Cancer (AJCC) 2009 final guidelines (Balch et al., 2009, Final version of 2009 AJCC melanoma staging and classification. Journal of Clinical Oncology). Staging combines several parameters, including mitotic index, tumor thickness, ulceration status, nodal status, and metastasis.
b. Staging in the methods of the invention can be incorporated as a continuous variable (stage 1, 2, 3, or 4).
Methods Based on Measuring the Level of miR-150-5p to Quantify Tumor-Infiltrating Lymphocytes (TILs)

As disclosed herein, miR-150-5p can be useful for quantifying lymphocytic burden (tumor-infiltrating lymphocytes (TILs)) in tumors that expresses low levels or do not express miR-150 (e.g., melanoma, prostate cancer, breast cancer, hepatocellular carcinoma, renal cell carcinoma, colorectal carcinoma). miR-150 levels are likely more sensitive and quantitative than histological quantification of TILs (i.e., pathologist counting from H&E, Immunohistochemistry, or in situ stained sections). TIL response is not currently integrated into the staging guidelines for melanoma, but there is evidence that TIL response in primary melanoma correlates with Sentinel Lymph Node (SLN) status, disease progression, and patient outcomes (Azimi et al., 2012, J Clin Oncol, 30:2678-83; Grotz et al., 2013, Melanoma Research, 23:132-7; Taylor et al., 2007, J Clin Oncol, 25: 869-75).

In one embodiment of the TIL quantification method of the present invention, tumor tissue is dissected (e.g., using macrodissection or laser-capture microdissection) at the tumor border prior to RNA extraction.

In one specific embodiment, miR-150-5p is measured in RNA extracted from a defined number of lymphocytes from a patient's peripheral blood. Based on the measured level of miR-150-5p, an assessment can be made as to the approximate number of TILs present in a defined tumor area (e.g., using the percentage of TILs to tumor area).

In a separate embodiment, the invention provides a method for quantifying lymphocyte response to a tumor comprising measuring the level of miR-150-5p. In one specific embodiment of this method, the level of miR-150-5p is determined in RNA extracted from tumor and is compared to the level of miR-150-5p in the surrounding stroma.

In addition to the calculation parameters described above, in the method for quantifying lymphocytic burden in tumors based on miR-150-5p, titration of lymphocyte RNA can be used as a comparator (e.g., RNA can be extracted for a set number of lymphocytes and titration performed to understand miR-150-5p copy number per lymphocyte; alternatively or in parallel, artificial miR-150-5p mimics can be used to establish a standard curve of miR-150-5p molecules). Non-limiting examples of such comparator lymphocyte RNA include RNA from the patient's own lymphocytes or RNA extracted from lymphocytes of many individuals. Batches need to be qualified to ensure run-to-run continuity.

Thus, in one embodiment, the invention provides a method for predicting the likelihood of developing melanoma brain metastasis (B-Met) in a subject diagnosed with primary melanoma, said method comprising:

a. determining the levels of miR-150-5p, miR-15b-5p, miR-16-5p, and miR-374b-3p in a primary melanoma sample collected from the subject;

b. calculating a B-Met prognostic score based on a weighted summation of the miRNA levels determined in step (a) and melanoma stage of the primary melanoma sample collected from the subject;

c. comparing the B-Met prognostic score calculated in step (b) with a corresponding control value;

d. (i) identifying the subject as being at high risk of developing B-Met if the B-Met prognostic score calculated in step (b) is higher than the corresponding control value or (ii) identifying the subject as being at low risk of developing B-Met if the B-Met prognostic score calculated in step (b) is same or lower than the corresponding control value.

Non-limiting examples of the methods for determining the levels of the miRNA in the above method of the invention include, e.g., hybridization, array-based assays, RT-PCR-based assays, and sequencing.

In a further embodiment, the invention provides a method for predicting the likelihood of developing melanoma brain metastasis (B-Met) in a subject diagnosed with primary melanoma, said method comprising:

a. determining the levels of miR-150-5p, miR-15b-5p, miR-16-5p, and miR-374b-3p in a primary melanoma sample collected from the subject by an RT-PCR-based assay;

b. calculating a B-Met prognostic score based on a weighted summation of the miRNA levels determined in step (a) and melanoma stage of the primary melanoma sample collected from the subject using the formula

Score=−0.51 miR-150+0.65 miR15b−0.96 miR-16−0.24 miR-374+0.47*stage;

c. comparing the B-Met prognostic score calculated in step (b) or the B-Met prognostic probability calculated in step (c) with a corresponding control value;

d. (i) identifying the subject as being at high risk of developing B-Met if the B-Met prognostic score calculated in step (b) is higher than the corresponding control value or (ii) identifying the subject as being at low risk of developing B-Met if the B-Met prognostic score calculated in step (b) is same or lower than the corresponding control value.

In another embodiment, the invention provides a method for predicting the likelihood of developing melanoma brain metastasis (B-Met) in a subject diagnosed with primary melanoma, said method comprising:

a. determining the levels of miR-150-5p, miR-15b-5p, miR-16-5p, and miR-374b-3p in a primary melanoma sample collected from the subject by a microarray-based assay;

b. calculating a B-Met prognostic score based on a weighted summation of the miRNA levels determined in step (a) and melanoma stage of the primary melanoma sample collected from the subject using the formula

Score=−0.51 miR-150+0.65 miR15b−0.96 miR-16−0.24 miR-374+0.47*stage

c. comparing the B-Met prognostic score calculated in step (b) with a corresponding control value;

d. (i) identifying the subject as being at high risk of developing B-Met if the B-Met prognostic score calculated in step (b) is higher than the corresponding control value or (ii) identifying the subject as being at low risk of developing B-Met if the B-Met prognostic score calculated in step (b) is same or lower than the corresponding control value.

In one embodiment of any of the above methods, if there is more than one sample in the measured cohort, all miRNAs are standardized into mean 0 and unit variance variables by subtracting the mean and dividing by the standard deviation (SD) of the cohort, before fitting into the weighted summation formula.

Preferably, the methods of the invention are performed on patients diagnosed with stage I, II, or III primary cutaneous melanoma. In some embodiments, the methods specifically exclude patients diagnosed with non-cutaneous melanoma, such as uveal melanoma.

The methods of the invention make possible early prognosis of the likelihood of developing melanoma brain metastasis (B-Met), e.g., at the time of the initial melanoma diagnosis, allowing for better monitoring and early application of treatments to prevent or delay the occurrence of B-Met. Patients determined by the methods of the invention to be at high risk of developing B-Met will be likely subject to:

a. a more frequent (e.g., every 3 months) and detailed surveillance (e.g., involving brain imaging by MRI, CT, PET or other imaging modality) to identify B-Met;
b. more extensive primary tumor staging (e.g., involving sentinel lymph node biopsy (SLNB) and/or regional lymph node mapping), and
c. adjuvant treatment, e.g., using one or more of the following:
i. Interferon alpha (e.g., pegylated IFN NCT01502696),
ii. BRAF and/or MEK inhibitors (e.g., vemurafenib [Zelboraf, NCT01667419], dabrafenib (BRAFi)+trametinib (MEKi) [NCT01682083]),
iii. Immunotherapy (e.g., anti-CTLA4 [e.g., Ipilumimab [Yervoy, NCT00636168, NCT01274338], anti-PD1 [e.g., Nivolumab, MK-3475/Lambrolizumab, Pidilizumab, AMP-224], anti-PD-Li [e.g., MEDI-4736, MPDL3280A], Interleukin 2 [IL-2], Aldesleukin [Proleukin]);
iv. imatinib, nilotinib, dacarbazine, temozolomide,
v. Prophylactic cranial irradiation (PCI).

The method of the invention also allows for more precise identification of various groups of patients who can be then recruited in clinical trials to develop and/or test new treatments to prevent or treat B-Met.

### Treatment Methods of the Invention

Cellular pathways regulated by the prognostic miRNAs identified herein are potential molecular therapeutic targets for prevention and treatment of B-Met.

Thus, in one embodiment, the present invention also provides a method for prevention or treatment of melanoma brain metastasis (B-Met) in a subject in need thereof comprising increasing the level and/or activity of one or more miRNA selected from the group consisting of miR-150-5p, miR-16-5p, and miR-374b-3p in the melanoma cells of the subject. Such increase in the level and/or activity of said miRNAs can be achieved using any method known in the art (e.g., over-expressing miRNA or mature miRNA mimic [an oligonucleotide, usually with some structural change(s), of the same sequence as the mature miRNA], e.g., using viral constructs; using sense-based oligonucleotides or modified-oligonucleotide mimics [e.g., technologies from miRNA Therapeutics and miRagen Therapeutics]; inhibiting negative or activating positive miRNA regulators [transcriptional or epigenetic], etc.).

In another embodiment, the invention also provides a method for prevention or treatment of B-Met in a subject in need thereof comprising decreasing the level and/or activity of miR-15b-5p in the melanoma cells of the subject. Such decrease in the level and/or activity of miR-15b-5p can be achieved using any method known in the art (e.g., RNAi molecules or antisense oligonucleotides [e.g., Exiqon miRCURY LNA inhibitors, Dharmacon MiRIDIAN miRNA inhibitors, Santaris Tiny LNA]).

The methods of the invention involve measuring miRNA levels in cutaneous melanoma tissue samples. To achieve effective miRNA extraction, primary melanoma tissue can be either used fresh or frozen or can be processed (e.g., formalin-fixed paraffin-embedded (FFPE)). To improve reproducibility of preparations, the following non-limiting measures can be adopted:

i. Reduction of air-exposure-related RNA degradation (e.g., storage of sections in an inert air environment, performing RNA extraction within 3-4 days from the time of tissue sectioning to minimize air-related RNA degradation, or minimizing time to tissue fixation);
ii. Use of approximately 10×5 μm sections for RNA extraction;
iii. Use of tissue sections affixed to laser capture microdissection slides (such as, e.g., PENmembrane 2.0 slides (Leica)) or uncharged slides;
iv. Use of tumor tissue marked by a pathologist on an H&E-stained consecutive 5 μm tissue sections to exclude surrounding stroma during tissue dissection and ensure that extracted RNA is mostly derived from melanoma cells (preferably, >80% of tissue used for extraction should be of melanoma origin).

In some embodiments, prior to miRNA quantification, cutaneous melanoma tissue material is processed to isolate and purify total RNA or RNA enriched for miRNA. Useful methods of miRNA isolation and purification, include, e.g., Qiazol or Trizol extraction or the use of commercial kits (e.g., miRNeasy kit [Qiagen], MirVana RNA isolation kit [Ambion/ABI], miRACLE [Agilent], High Pure miRNA isolation kit [Roche], and miRNA Purification kit [Norgen Biotek Corp.]), concentration and purification on anion-exchangers, magnetic beads covered by RNA-binding substances, or adsorption of certain miRNA on complementary oligonucleotides.

In some embodiments, miRNA degradation in cutaneous melanoma tissue samples and/or during miRNA purification is reduced or eliminated. Useful methods for reducing or eliminating miRNA degradation include, without limitation, adding RNase inhibitors (e.g., RNasin Plus [Promega], SUPERase-In [ABI], etc.), use of guanidine chloride, guanidine isothiocyanate, N-lauroylsarcosine, sodium dodecylsulphate (SDS), or a combination thereof. Reducing miRNA degradation in samples is particularly important when sample storage and transportation is required prior to miRNA quantification.

Non-limiting examples of useful methods for measuring miRNA level in cutaneous melanoma tissue samples include:

a. polymerase chain reaction (PCR)-based detection, such as, e.g., stem-loop or poly-A-based reverse transcription-polymerase chain reaction [RT-PCR] (non-limiting examples of useful commercial assays include Taqman miRNA assays [stem-loop assays; Applied Biosystems] and LNA-based miRNA PCR assays [poly-A-based assays; Exiqon]) or quantitative RT-PCR based array method (qPCR-array);
b. hybridization with selective probes, such as, e.g., using Northern blotting, bead-based flow-cytometry, miRNA or oligonucleotide microchip [microarray] (e.g., commercial arrays from Agilent, Exiqon, Affymetrix or custom-designed two-color arrays with a common reference [e.g., a specific quantity of ‘artificial’ miRNA for all probes on the chip or a specific sample such as, e.g., large batches of RNA isolated from patient blood, etc]), or solution hybridization assays such as Ambion mirVana miRNA Detection Kit), and
c. direct sequencing by one of the next generation sequencing technologies (e.g., Helicos small RNA sequencing, miRNA BeadArray (Illumina), Roche 454 (FLX-Titanium), and ABI SOLiD). For review of additional applicable techniques see, e.g., Chen et al., BMC Genomics, 2009, 10:407; Kong et al., J Cell Physiol. 2009; 218:22-25.

### Kits of the Invention

In conjunction with the prognostic methods, the present invention also provides various kits comprising primer and/or probe sets specific for the detection of biomarker miRNAs miR-150-5p, miR-15b-5p, miR-16-5p, and miR-374b-3p (or any subset thereof).

The kits of the invention can be useful for direct miRNA detection in primary melanoma tumor samples isolated from patients or can be used on purified miRNA samples.

A kit of the invention can also provide reagents for primer extension and amplification reactions. For example, in some embodiments, the kit may further include one or more of the following components: a reverse transcriptase enzyme, a DNA polymerase enzyme (such as, e.g., a thermostable DNA polymerase), a polymerase chain reaction buffer, a reverse transcription buffer, and deoxynucleoside triphosphates (dNTPs). Alternatively (or in addition), a kit can include reagents for performing a hybridization assay. The detecting agents can include nucleotide analogs and/or a labeling moiety, e.g., directly detectable moiety such as a fluorophore (fluorochrome) or a radioactive isotope, or indirectly detectable moiety, such as a member of a binding pair, such as biotin, or an enzyme capable of catalyzing a non-soluble colorimetric or luminometric reaction. In addition, the kit may further include at least one container containing reagents for detection of electrophoresed nucleic acids. Such reagents include those which directly detect nucleic acids, such as fluorescent intercalating agent or silver staining reagents, or those reagents directed at detecting labeled nucleic acids, such as, but not limited to, ECL reagents. A kit can further include miRNA isolation or purification means as well as positive and negative controls. A kit can also include a notice associated therewith in a form prescribed by a governmental agency regulating the manufacture, use or sale of diagnostic kits. Detailed instructions for use, storage and troubleshooting may also be provided with the kit. A kit can also be optionally provided in a suitable housing that is preferably useful for robotic handling in a high throughput setting.

The components of the kit may be provided as dried powder(s). When reagents and/or components are provided as a dry powder, the powder can be reconstituted by the addition of a suitable solvent. It is envisioned that the solvent may also be provided in another container. The container will generally include at least one vial, test tube, flask, bottle, syringe, and/or other container means, into which the solvent is placed, optionally aliquoted. The kits may also comprise a second container means for containing a sterile, pharmaceutically acceptable buffer and/or other solvent.

Where there is more than one component in the kit, the kit also will generally contain a second, third, or other additional container into which the additional components may be separately placed. However, various combinations of components may be comprised in a container.

Such kits may also include components that preserve or maintain DNA or RNA, such as reagents that protect against nucleic acid degradation. Such components may be nuclease or RNase-free or protect against RNases, for example. Any of the compositions or reagents described herein may be components in a kit.

### EXAMPLES

The present invention is also described and demonstrated by way of the following examples. However, the use of these and other examples anywhere in the specification is illustrative only and in no way limits the scope and meaning of the invention or of any exemplified term. Likewise, the invention is not limited to any particular preferred embodiments described here. Indeed, many modifications and variations of the invention may be apparent to those skilled in the art upon reading this specification, and such variations can be made without departing from the invention in spirit or in scope. The invention is therefore to be limited only by the terms of the appended claims along with the full scope of equivalents to which those claims are entitled.

### Example 1

### Materials and Methods

Study Population and Clinical Samples

All patients in this study were enrolled in the Interdisciplinary Melanoma Cooperative Group (IMCG) database of NYU Langone Medical Center (NY, N.Y.). Informed consent was obtained from all patients and approval acquired by the Institutional Review Board of NYU School of Medicine (protocol #10362). Primary human melanoma samples were collected at the time of surgery (Training cohort: 1989:2007, Validation cohort: 1994:2009, Independent cohort: 1997:2010). IMCG patients were actively, prospectively followed up every 6 months after primary tumor removal and every 3 months after a recurrence. Site of metastasis and date of diagnosis is annotated in the IMCG database at the time of such diagnoses. Parameters relevant to this study include date of diagnosis, Breslow thickness, ulceration presence, mitotic index, and stage for primary tumors; dates of diagnoses and anatomical locations of metastases, date of last follow-up or death, last clinical status, and cause of death. Patients were selected using the following criteria: For the initial study cohort (training), we excluded patients who were non-recurrent/metastatic and who were not expected to reach >3 years of follow-up or death by study end. We excluded all non-cutaneous melanoma patients. From the remaining cases, we randomly selected 92 cases, with a ratio of half non-recurrent to half recurrent patients. Within recurrent patients, to maximize the chance to detect a prognostic signature of the development of brain metastasis, we selected approximately 50% who had already developed brain metastasis. Subsequent validation cohorts were selected similarly. All patients were treated surgically for removal of primary melanoma lesions. All patients developing B-Met did so during active follow up. Clinical status at last date of follow-up is recorded as “alive, no melanoma,” “alive with melanoma,” “died with melanoma,” “died, no melanoma,” or “died, cause unknown.” Medical oncologists reviewed all deceased patients' histories and last clinical statuses to determine if melanoma was the cause of death. All tumors were classified according to the 2009 American Joint Committee on Cancer (AJCC) staging system (Balch et al. 2009, Final version of 2009 AJCC melanoma staging and classification. Journal of Clinical Oncology).

Clinical Specimens

All melanoma specimens were human primary, cutaneous melanoma samples that were collected, formalin fixed, and paraffin embedded at the time of surgery (Discovery cohort: 1989:2007, Validation cohort: 1994:2009, Independent cohort: 1997:2010). All tumors were classified according to the 2009 American Joint Committee on Cancer (AJCC) staging system.

RNA Extraction

RNA was extracted from macro-dissected formalin-fixed, paraffin embedded (FFPE) tissue sections using miRNeasy FFPE kit (Qiagen), following manufacturer's recommendations. 5 μm sections of FFPE samples (3-12 sections per patient depending on the size of the primary tumor) were attached to PEN-Membrane 2.0 μm slides (Leica) designed for laser capture micro-dissection. Primary melanoma tissues were macroscopically dissected using disposable scalpels (Feather No. 11) under a dissecting microscope and guided by images of hematoxylin and eosin (H&E) staining of consecutive sections on which a board-certified pathologist marked areas of tumor. Exclusion of surrounding stroma by macro-dissection consistently allowed RNA extraction from greater than 80% tumor.

miRNA Microarray Expression Profiling and Data Processing

miRNA expression profiling of FFPE-extracted RNA from primary melanomas was performed by Exiqon, Inc. Briefly, a reference sample was generated by mixing an equal amount of all samples analyzed, independently for each cohort. RNA from sample and reference were labeled with Hy3™ or Hy5™ fluorescent label, respectively, mixed pair-wise, and hybridized to the miRCURY™ LNA arrays (Exiqon, Denmark). Arrays were scanned using the Agilent G2565BA Microarray Scanner System (Agilent Technologies, Inc., USA). Image analysis was carried out using the ImaGene software (BioDiscovery, Inc., USA). The quantified signals were background corrected (Normexp with offset value 10) and normalized using the global Lowess (LOcally WEighted Scatterplot Smoothing) regression algorithm. Data were represented as the Log 2 transformation of the ratio of median intensities of Hy3-labeled sample to Hy5-labeled reference. Subsequent analyses were performed with R 2.14.0.

microRNA Real-Time qPCR and Data Processing

10 ng RNA was reverse transcribed in 10 μl reactions using the miRCURY LNA™ Universal RT microRNA PCR, Polyadenylation and cDNA synthesis kit (Exiqon, Denmark). cDNA was diluted 50× and assayed in 10 μl PCR reactions according to the protocol for miRCURY LNA™ Universal RT microRNA PCR; each miRNA was assayed once by qPCR on the microRNA Ready-to-Use PCR, Human pick-n-mix panel (Exiqon). Negative controls excluding template from the reverse transcription reaction were performed and profiled like the samples. The amplification was performed in a LightCycler® 480 Real-Time PCR System (Roche, Germany) in 384 well plates. The amplification curves were analyzed using the Roche LC software, both for determination of crossing point/threshold (Cp or Ct) (by the 2nd derivative method) and for melting curve analysis. The amplification efficiency was calculated using algorithms similar to the LinReg software (Exiqon). All assays were inspected for distinct melting curves and the Tm was checked to be within known specifications for the assay. Furthermore, assays must be detected with 5 Cp's less than the negative control, and with Cp<37 to be included in the data analysis. Data that did not pass these criteria were omitted from any further analysis. Using NormFinder (Andersen et al., Cancer Res 2004; 64:5245-50) three miRNA were identified to be most stably expressed across all samples (hsa-let7e-5p, miR-30d-5p, and miR-423-3p). The average Cp for these miRNA was employed as a normalizer for this data set. A normalized Cp was defined as NormCp=Cp (average of 3 normalizers)−Cp (sample).

Quality Control of microRNA qPCR

cDNA were generated for each RNA sample in duplicate reverse transcription (RT) reactions and tested for the expression of 5 miRNA (miR-103, miR-191, miR-23a, miR-30c, and miR-451) and a synthetic spike-in RNA (UniSp6). An average Cp was calculated for the duplicate RTs, and evaluation of expression levels was performed based on raw Cp values. All five miRNA were detected in most samples, with a few samples showing less expression of individual miRNA. No RT-qPCR inhibition occurred as judged by the expression of the UniSp6 spike-in.

Evaluation of Tumor Infiltrating Lymphocytes (TILs) by Histological Analysis of Primary Tumors

The presence or absence of tumor-infiltrating lymphocytes (TILs) was recorded. The cases with TILs were further sub-classified into brisk and non-brisk based on the published criteria (Clark et al., Journal of the National Cancer Institute 1989, 81:1893-904; Clemente et al., Cancer 1996, 77:1303-10). Two-sample unpaired t test was used to compare log transformed miRNA expression between brisk and non-brisk TILs. The association between brisk status and B-Met was assessed by χ² test.

CD45 Immunohistochemistry Staining

Immunohistochemistry was performed on 5 micron formalin fixed, paraffin embedded (FFPE) primary melanoma tissue using mouse anti-human CD45, (Leukocyte Common Antigen, LCA), clone RP2/18 (Cat. 760-2505, Ventana Medical Systems Tucson, Ariz. USA). Sections were deparaffinized in xylene (3 changes), rehydrated through graded alcohols (3 changes 100% ethanol, 3 changes 95% ethanol) and rinsed in distilled water. Antibody incubation and detection were carried out at 40° C. on a NexES instrument (Ventana Medical Systems Tucson, Ariz. USA) using Ventana's reagent buffer and detection kits unless otherwise noted. Endogenous peroxidase activity was blocked with hydrogen peroxide. CD45 was applied neat (undiluted) as directed by the manufacturer and incubated for 30 minutes. Primary antibody was detected with iView biotinylated goat anti-mouse followed by application of streptavidin-horseradish-peroxidase conjugate. The complex was visualized with 3,3 diaminobenzidene and enhanced with copper sulfate. Slides were washed in distilled water, counterstained with hematoxylin, dehydrated and mounted with permanent media. Appropriate positive and negative controls were included with the study sections. An attending pathologist who was blinded to patients' clinical data scored CD45 expression as the absolute number of positively-stained immune cells demonstrating characteristic lymphocytic morphology in a representative high-power field (HPF; 0.2 mm²) that was selected by scanning each slide at ×40 to find the field with the highest antibody expression. The CD45 expression was assessed in two locations: intratumoral and peritumoral (within 0.5 mm from the tumor edge) by an attending pathologist blinded to patient information. In addition, the percentage of CD45-positive cells, defined as the ratio of CD45-positive cells over tumor cells in one high-power field, was calculated in the intratumoral location. Pearson correlation coefficients were used to characterize the correlation between log transformed miRNA expression and CD45 expression in the three separate locations.

Statistical Analyses

Using the discovery cohort (n=92), we analyzed results for 1,360 miRNA, of which data for 578 miRNA were available for at least 67% samples. All miRNA were standardized into mean 0 and unit variance variables by subtracting the mean and dividing by the standard deviation (SD) within each cohort for scale consistency across cohorts. miRNAs were first ranked by univariate association of expression level for each miRNA with brain metastasis-free survival via Cox proportional hazards regression analysis with adjustment for tumor stage as a continuous variable. The top 50 ranking miRNAs were used as candidates included in the multivariate Cox proportional hazards model with tumor stage in the regression. The 4 miRNA-signature was selected by minimizing Akaike's information criterion (AIC) of the multivariate Cox proportional hazards model through forward stepwise selections (Klein et al., Statistics for biology and health, 1997 xiv, p. 502.) The linear combination of model predictors weighted by regression coefficients was defined as the risk score. To test the classifier, regression coefficients of the Cox model were applied to the validation cohort and the independent cohort to obtain their risk scores. The discriminating power of the risk score was evaluated by Harrell's C index (Harrell et al., JAMA 1982, 247:2543-6) in the three cohorts separately. The risk scores were also evaluated for utility in predicting brain metastasis by identifying the area under the Receiver Operating Characteristic (ROC) curve (AUC) in the three cohorts with ≧3 years brain metastasis-free survivals separately (Wang et al., Biometrics 2011, 67: 896-905). For this purpose, brain metastasis patients were defined as cases and non-brain metastasis patients (non-recurrent/non-metastatic patients and recurrent but non-brain metastatic patients) with ≧3 years of follow-up were defined as controls. A risk score cutoff using the Youden Index of the ROC curve was chosen to separate patients into high- and low-recurrence risk groups (Youden W. J., Cancer 1950, 3: 32-5). The same cutoff was applied to the validation and independent cohorts. Kaplan-Meier survival curves and Wilcoxon tests, as it is more robust than log-rank tests to non-consistent hazard ratios (Harrington and Fleming, Biometrika 1982, 69:553-566), were used to compare the brain metastasis-free survival and overall survival distributions of the two groups in each cohort.

Kruskal-Wallis rank sum test was used to compare the risk scores among non-recurrent/non-metastatic, non-brain metastatic, brain metastatic concomitant with or subsequent to other sites, and isolated, first-site brain metastatic patient groups. Spearman correlation coefficient was used to assess the concordance between the score correlations of the same set of patients measured under different platforms. All statistical analyses were performed in R 2.14.0.

To assess the robustness of the miRNA signature score under various technology platforms, the miRNA expression values were measured via miRCURY LNA™ Universal RT microRNA PCR as described (Exiqon) on 48 and 62 specimens who had been also measured by miRNA array in the discovery cohort and validation cohort, respectively. The miRNA were first standardized into mean 0 and unit variance variables for scale consistency across platforms. Regression coefficients of the logistic regression model, developed from the discovery cohort array measurements, were applied to obtain their risk scores. Spearman correlation coefficient was used to assess the concordance between the score correlations of the same set of patients measured under different platforms. All statistical analyses were performed in R 2.14.0.

### Results

### Brain-Metastasis Prognostic miRNA Signature Derived from Primary Melanoma Tissue

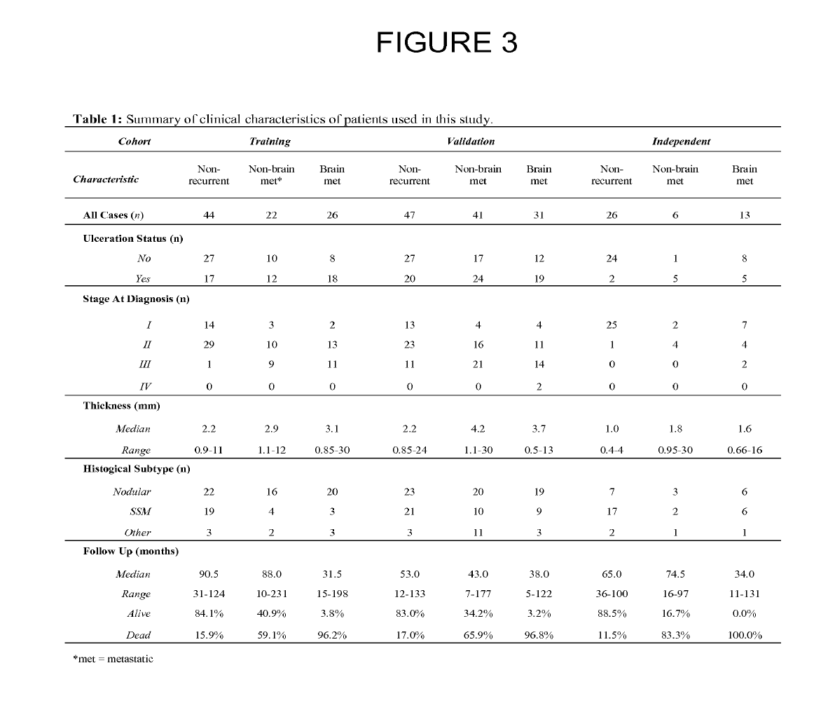

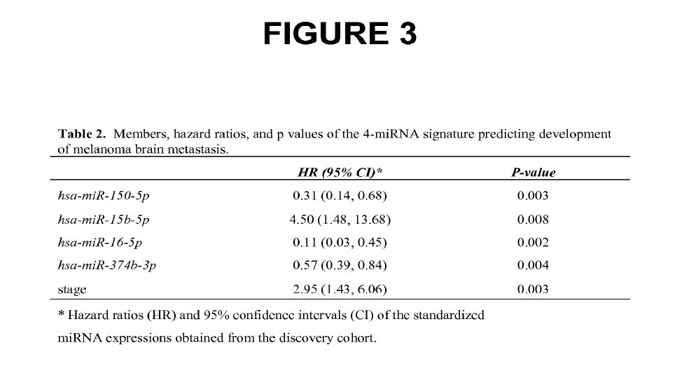

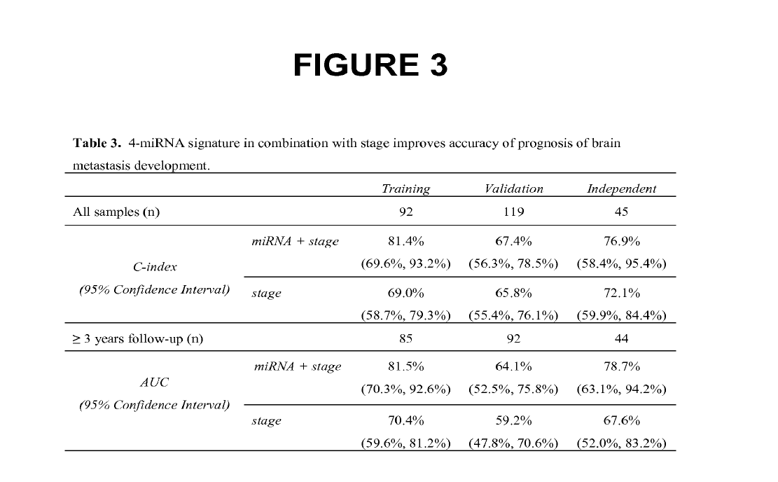

To explore the potential prognostic value of microRNA in primary melanoma, microRNA expression profiling was performed (miRCURY™ LNA arrays; Exiqon, Denmark) on total RNA extracted from formalin-fixed paraffin-embedded (FFPE) primary melanoma tissues. In contrast to messenger RNA, microRNA expression levels and patterns are highly stable in FFPE tissue, with expression comparable to that of fresh tissue (Bovell et al., Front. Biosci. 2012, 4: 1937-40, Hall et al., Br J Cancer 2012, 107: 684-94, Peiro-Chova et al., Virchows Arch 2013, 463: 765-74, Xi et al., RNA 2007, 13: 1668-74). All patients were enrolled in New York University's (NYU) Interdisciplinary Melanoma Cooperative Group (IMCG) database and were selected using the following criteria: For the initial study cohort (training), patients who were non-recurrent/metastatic and who would not reach >3 years of follow-up or death by study end were excluded. From the remaining cases, 92 cases were randomly selected, with a ratio of half non-recurrent to half recurrent patients. Within recurrent patients, to maximize the chance to detect a prognostic signature of the development of brain metastasis, approximately 50% were selected who had already developed brain metastasis. Subsequent validation cohorts were selected similarly. FIG. 3A presents a summary of clinicopathological features of patient groups included in this study. Using miRNA expression profiling data from our training cohort, a prognostic model of the development of brain metastasis was established. For that, the top-ranking miRNAs were identified by univariate association of expression with brain metastasis-free survival via Cox proportional hazards (PH) regression analysis with adjustment for tumor stage. This candidate set was used in multivariate Cox PH regression analysis, including tumor stage coded as a continuous variable, to establish a model predictive of brain metastasis-free survival. This analysis revealed a 4-miRNA signature (miR-15b-5p, miR-150-5p, miR-16-5p, and miR-374b-3p) with adjustment for tumor stage (FIG. 3B) that robustly predicts brain metastasis development (Harrell's C-index of 81.4% (95% CI=69.6%, 93.2%). In contrast, a model using clinical stage alone achieved Harrell's C-index of 69.0%.

From the training cohort, risk scores for individual patients were defined as the linear combination of the 4 miRNAs and tumor stage weighted by their regression coefficients in the Cox model. The same set of coefficients was applied to microarray or targeted RT-qPCR data, respectively, of two additional patient cohorts (validation (n=119) and independent (n=45)) to obtain risk scores. In combination with stage, this brain metastasis-distinguishing miRNA classifier displayed Harrell's C-index of 67.4% (95% CI=(56.3%, 78.5%)) and 76.9% (95% CI=(58.4%, 95.4%) in the validation and independent cohorts cohort respectively (FIG. 3C). Using the same patient cohorts, a model using clinical stage alone achieved Harrell's C-index of 65.8% and 72.1%, respectively.

ROC Curves Supportive of Prognostic Potential of miRNA Signature

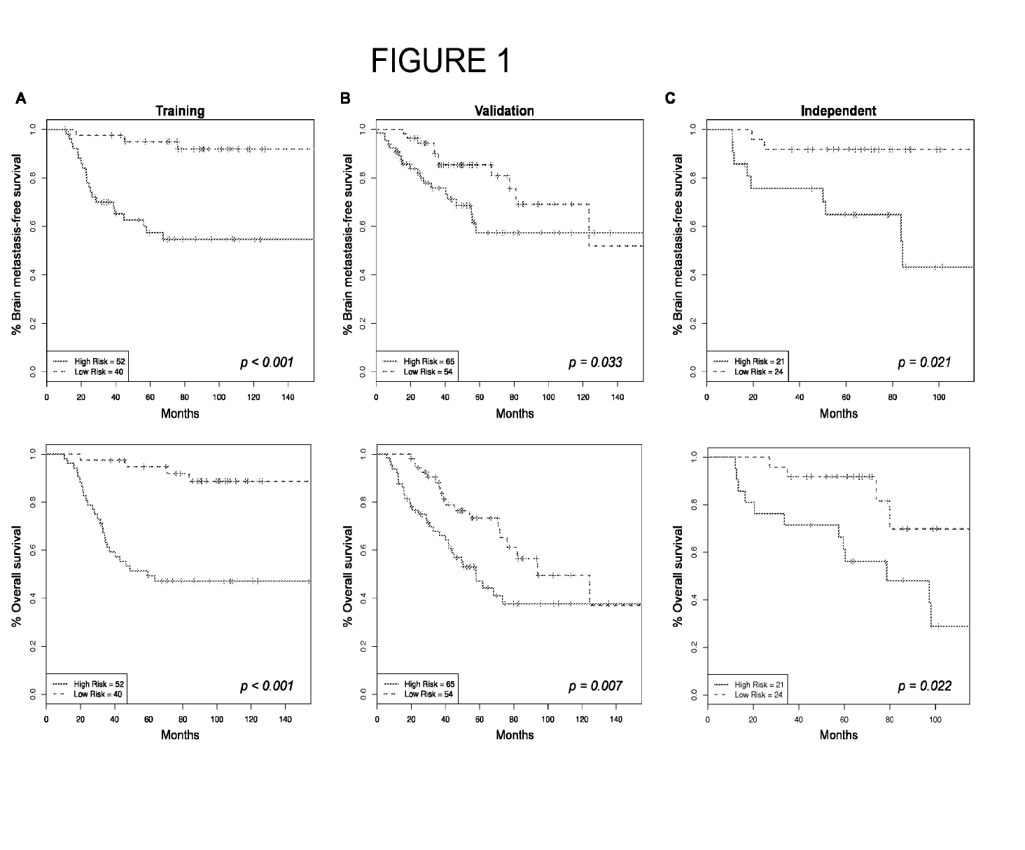

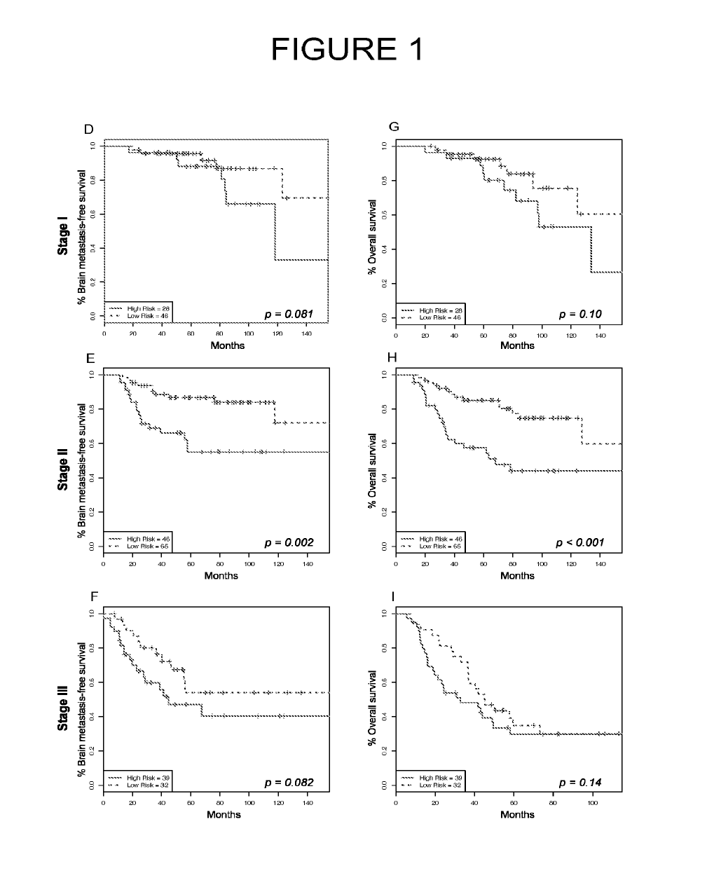

To further evaluate the prognostic performance of the miRNA signature obtained by Cox analysis, the area under the curve (AUC) of the receiver operating characteristic (ROC) curves for development of brain metastasis was calculated. For this purpose, brain metastasis patients were used as cases (n=26, 31, 13) and non brain-metastasis patients with >3 years of follow-up since initial melanoma diagnosis were used as controls (n=59, 61, 31). In combination with stage, this brain metastasis-distinguishing miRNA classifier displayed an AUC of 81.5% (95% CI=(70.3%, 92.6%)), 64.1% (95% CI=(52.5%, 75.8%)) and 78.7% (95% CI=(63.1%, 94.2%) in the training, validation and independent cohorts, respectively (FIG. 3C). In contrast, using the same patient cohorts, a model using clinical stage alone yielded AUCs of 70.4% (95% CI=(59.6%, 81.2%), 59.2% (95% CI=(47.8% 70.6%) and 67.6% (95% CI=(52.0%, 83.2%), respectively. The Youden index of the ROC curve of the discovery cohort was selected as a risk-score cutoff to stratify patients into high- and low-risk groups. For patients predicted to be negative for brain metastasis (below the cutoff) by the miRNA signature in the three cohorts (negative predictive value), 87.5%, 75.0%, and 87.5% patients have not developed brain metastasis after ≧3 years follow-up since initial melanoma diagnosis, supporting the potential clinical usage of the signature. High- and low-risk groups plotted in Kaplan-Meier survival curves displayed significant differences in the three patient cohorts for brain-metastasis-free survival (p<0.001, p=0.033, and p=0.021, respectively) and overall survival (p<0.001, p=0.007, and p=0.022, respectively) (FIG. 1A).

Finally, to further demonstrate the prognostic value of the miRNA signature beyond stage alone, the signature's performance within individual at-diagnosis stages for each cohort was estimated. High- and low-risk groups plotted in Kaplan-Meier survival curves showed the best statification for patients with stage II tumors in the validation cohort (FIG. 1B). These results suggest that the miRNA signature would likely be more beneficial to melanoma patients whose tumors have not advanced to clinically evident nodal disease.

Collectively, these results demonstrate that expression of a small set of miRNA, measured from primary melanoma tissues at initial melanoma diagnosis, is a strong predictor of the development of brain metastasis.

miRNA Signature Reflects Brain-Specific Tropism for Some Melanomas

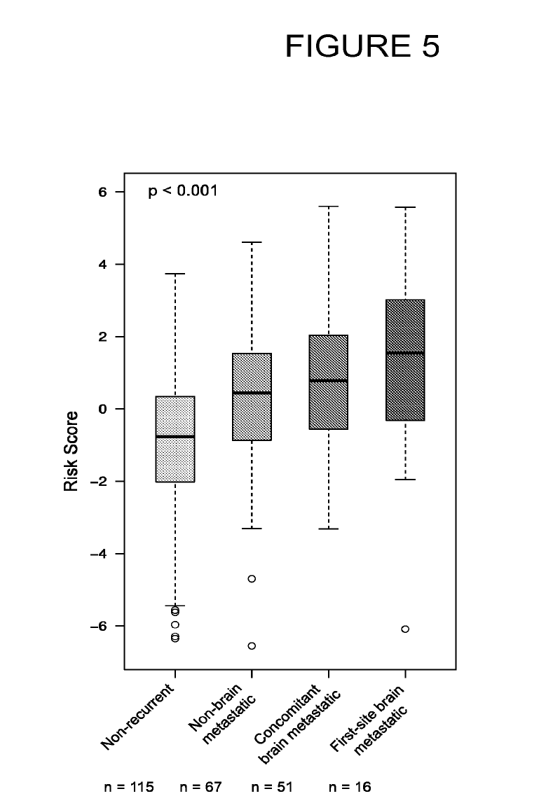

A subgroup of 36% of melanoma brain metastasis patients whose tumors spread to the brain as the isolated, first site of visceral metastasis were identified (Ma et al., Neuor Oncol 2012, 14: 849-58). This subset of patients suggests that some melanomas may be clinicopathologically and/or molecularly distinct and may develop molecular alterations that dictate tissue tropism of metastatic cells. To examine if the described miRNA signature is reflective of this possibility, the studied melanoma patients were further stratified into four groups: non-recurrent/non-metastatic (n=115), non-brain metastatic (n=67), brain metastasis concomitant with or subsequent to other sites of metastasis (n=51) and brain metastasis as the isolated, first site of visceral metastasis (n=16). The median signature risk scores reflect the difference among these four groups (p<0.001, Kruskal-Wallis rank sum test; FIG. 5). This sub-analysis suggests that the described signature may be reflective of brain-specific tropism for some melanomas.

miR-150-5p Levels Correlate with the Presence of Tumor Infiltrating Lymphocytes

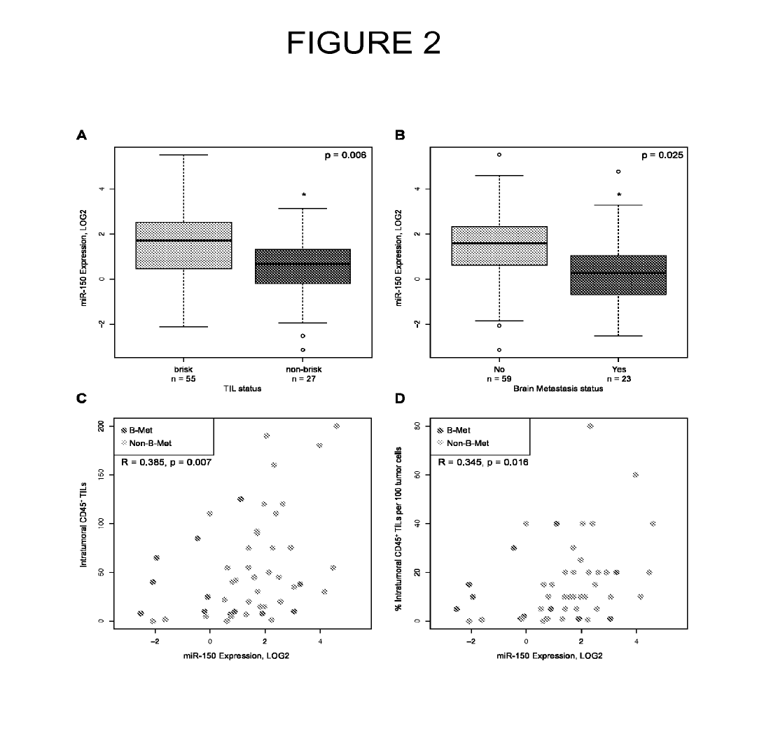

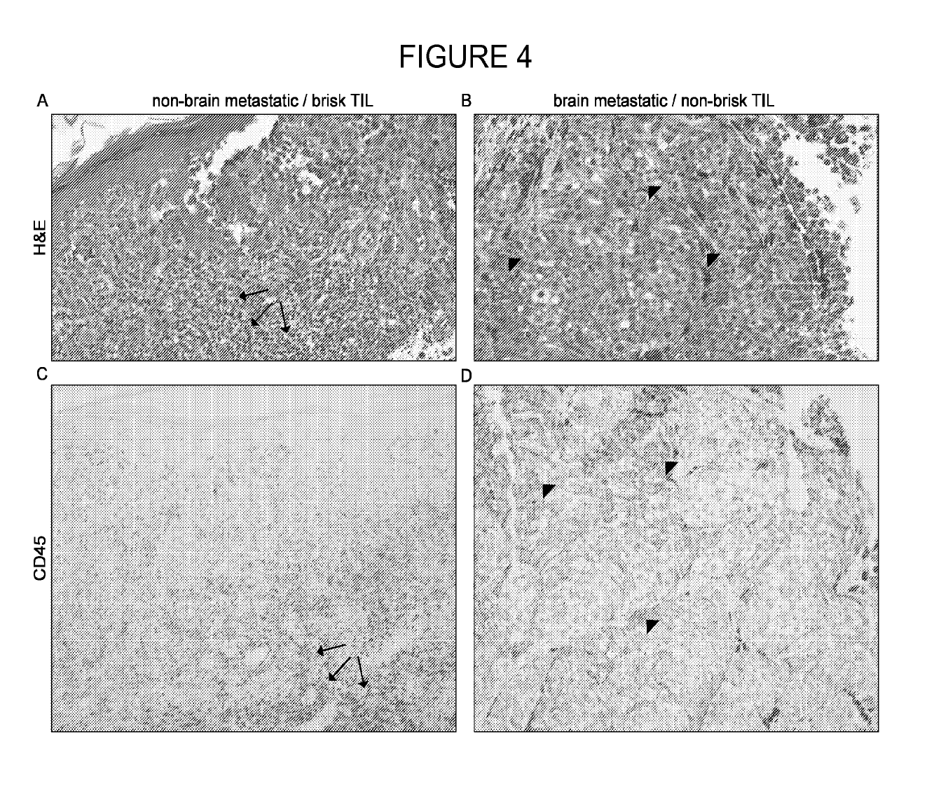

The prognostic value of miR-150-5p was further explored, because it is not expressed in melanoma cell lines and short-term cultures (unpublished data and (Stark et al., PLoS ONE 2010, 5: e9685), but well detected in melanoma tissues, which contain mixed cell-type populations. Since miR-150-5p is highly expressed in mature B and T-cells (Zhou et al., Proc Natl Acad Sci 2007, 104: 7080-5, Xiao et al., Cell 2007, 131: 146-59), it was hypothesized that differences in its expression between primary melanomas of different outcomes may be reflective of tumor infiltrating lymphocytes (TILs), rather than of a tumor-cell intrinsic property. Indeed, we found that miR-150-5p levels were significantly lower in samples classified as ‘non-brisk’ (n=27) compared to ‘brisk’ (n=55) TIL response (p=0.006, FIG. 2A) in a set of 82 primary melanoma samples (23 metastatic to brain, 20 metastatic to other sites, and 39 non-metastatic), with lower miR-150-5p detection associated to higher brain-metastasis occurrence (p=0.025, FIG. 2B). To independently confirm the correlation between miR-150-5p levels and hematopoietic cell infiltrates, 48 primary melanomas, which were previously analyzed for miR-150-5p expression and TILs, were stained for the leukocyte marker, CD45. It was found that miR-150-5p positively correlates with the absolute and relative number of intratumoral CD45⁺ cells (p=0.007 and p=0.016 respectively), but not with adjacent peritumoral CD45⁺ cells (data not shown) (FIG. 2C, D and FIG. 4). Collectively, these results indicate that miR-150-5p expression detected in primary melanoma tissues is likely to derive from TILs. Moreover, these data suggest that defective immune response to a primary melanoma or active immune evasion by melanoma cells may enable increased brain-metastasis potential for some patients' tumors.

### Discussion

The present Example discloses the prognostic capacity of miRNA expression of primary melanoma tissues, particularly their ability to predict development of brain metastasis. Using Cox regression analysis to establish a microRNA-based signature based on global miRNA expression patterns of primary melanoma samples from a cohort of 92 patients with extensive follow-up, a prognostic method is disclosed that, combines expression levels of 4 miRNA (miR-150-5p, miR-15b-5p, miR-16-5p, miR-374b-3p) with stage and robustly classifies patients by risk of melanoma brain metastasis. This method was verified in two additional, independent patient cohorts. Further, the present invention discloses that miR-150-5p, a lymphocyte regulator, is independently and inversely associated with melanoma brain metastasis and positively correlates with the presence of infiltrated primary tumors (TILs).

The above analyses identified a 4-microRNA (miR-150-5p, miR-15b-5p, miR-16-5p, and miR-374b-3p) prognostic signature that, in combination with stage, distinguished primary melanomas that metastasized to the brain from non-recurrent and non-brain-metastatic primary tumors (C-index=81.4%) in the training cohort. Application of this model to two validation cohorts yielded comparable results. Corresponding Kaplan-Meier curves of high-vs. low-risk patients displayed a clear separation in brain-metastasis-free and overall survival (training: p<0.001, p<0.001, validation: p=0.033, p=0.007, independent: p=0.021, p=0.022, respectively). Finally, of the microRNA in the prognostic model, the expression of a key lymphocyte miRNA, miR-150-5p, which is less abundant in primary melanomas metastatic to brain, was found to be correlated with presence of CD45+ tumor infiltrating lymphocytes.

Defining an accurate prognosis of melanoma patients, particularly those with early stage disease, remains difficult. Primary melanoma patient management is guided by gross histopathological criteria, which clinicians use to select patients for more extensive staging, including sentinel lymph node evaluation, intensified surveillance, and/or adjuvant therapy (e.g. interferon-alpha). However, low- and moderate-risk patients with histopathologically similar tumors can have vastly different outcomes, supporting the notion that current staging insufficiently captures patient/tumor heterogeneity. In supplement of histopathological features, molecular changes within tumors of similar staging hold immense promise to better understand, diagnose, prognosticate, and develop treatments for cancer.

The utility of miRNA as biomarkers of cancer outcomes has been increasingly explored due to their tissue/lineage specificity, ease of quantification, and stability in sera and processed tissues. Studies of a variety of cancers have identified associations between miRNA and clinical outcome (Yanaihara et al., Cancer Cell, 2006, 9:189-98; Takamizawa et al., Cancer Res., 2004, 64:3753-6; Schaefer et al., Int J Cancer, 2010, 126:1166-76; Brenner et al., World J Gastroenterol, 2011, 17:3976-85) but only a few have prognostic modeling (Yu et al., Cancer Cell, 2008, 13:48-57; De Preter et al., Clin Cancer Res, 2011, 17:7684-92.). Moreover, miRNA as prognostic biomarkers have not been rigorously explored in primary melanoma tissues (Pencheva et al., Cell, 2012, 151:1068-82; Gaziel-Sovran et al., Cancer Cell, 2011, 20:104-22; Satzger et al., Int J Cencer, 2010, 126:2553-615; Friedman et al., J Translat Med, 2012, 10:155.).

The current invention discloses that miRNAs can be used as prognostic biomarkers of site-specific metastasis (to the brain) in primary melanoma tissues. This supports a deterministic model of melanoma evolution, in which a tumor's progression is encoded early in its natural history (Scott et al., Cancer Cell, 2011, 20:92-103; Turajlic et al., Genome Res, 2012, 22:196-207; Segura et al., Clin Cancer Res, 2010, 16:1577-86.). In addition, this invention discloses that some molecular alterations in some primary melanomas may be reflective of brain-specific tropism as opposed to non-specific metastasis. Characterization of the studied melanoma patients into four groups: non-recurrent/non-metastatic (n=122), Non-B-Met-recurrent (n=66), B-Met concomitant with other sites of metastasis (n=51) and B-Met as the isolated first site of visceral metastasis (n=17) demonstrated that the signature risk score reflects the difference among the four groups (p<0.001, Kruskal-Wallis rank sum test, FIG. 5). This sub-analysis further supports that the method disclosed by the current invention is determinative of brain-specific tropism, determined at early stages of tumorigenesis, for some melanomas.

Of the miRNA used in this B-Met predictive method, the prognostic role for miR-150-5p is intriguing, because its expression seems to not be melanoma-cell intrinsic. In primary melanoma tissues, miR-150-5p was well detected and inversely correlated with brain metastasis in three patient cohorts. However, miR-150-5p is not detectable in isolated melanoma cultures (Stark et al., PLoS ONE 2010, 5: e9685). This paradox may be explained by a melanoma-cell extrinsic source for miR-150-5p detected in tissues. As miR-150-5p is highly expressed in and a key regulator of mature B and T-cells (Zhou et al., PNAS, 2007, 104:7080-5; Xiao et al., Cell, 2007, 131:146-59; Ghisi et al., Blood, 2011, 117:7053-62), the present inventors hypothesized that TILs may be the source of detected miR-150-5p. Indeed, as demonstrated herein, miR-150-5p levels strongly correlated with CD45⁺ lymphocytes that had infiltrated primary tumors (TILs), but not peritumoral CD45⁺ lymphocytes. The immune system, for some still unknown reasons, restrains some melanomas. The present findings strongly suggest that immune cells are key suppressors of melanoma progression generally and B-Met, specifically. In addition, the present invention demonstrates that miR-150-5p expression represents an alternative means of measuring TIL response.

A strong molecular classifier, such as that described here, capable of ascribing reliable risk of disease progression at the time of initial melanoma diagnosis provides a valuable tool to improve melanoma patient management. This invention is particularly important for prediction of brain metastasis, which causes the majority of deaths from melanoma. The present invention is especially relevant in light of the recent surge in melanoma treatment options for which ongoing clinical trials (Accession Nos. NCT01667419, NCT01274338, NCT01682213) will soon determine the usefulness of BRAF inhibitors (vemurafenib and dabrafenib) and anti-CTLA4 immunotherapy (ipilimumab) to prevent recurrence in the adjuvant setting. Moreover, these therapeutics have already been beneficial for some patients with B-Met (Falchook et al., Lancet 2012; 379:1893-901; Long et al., The Lancet Oncology 2012; 13:1087-95; Schartz et al., Melanoma Research 2010; 20:247-50) suggesting that treatment options efficacious in the adjuvant setting for high-risk primary melanoma patients may already be in use. Accurate stratification of patients by risk of developing B-Met would inform patient selection for such adjuvant treatments and/or be a rationale to explore prophylactic cranial irradiation (PCI) or other therapies for high-risk primary melanoma patients in future clinical trials. As importantly, the lowest risk patients could potentially be spared the associated morbidity of extensive nodal staging, adjuvant therapies, and interventional trials.

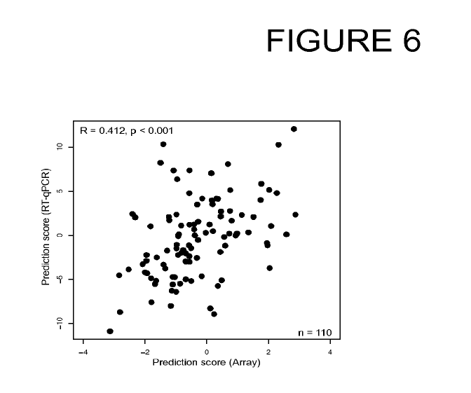

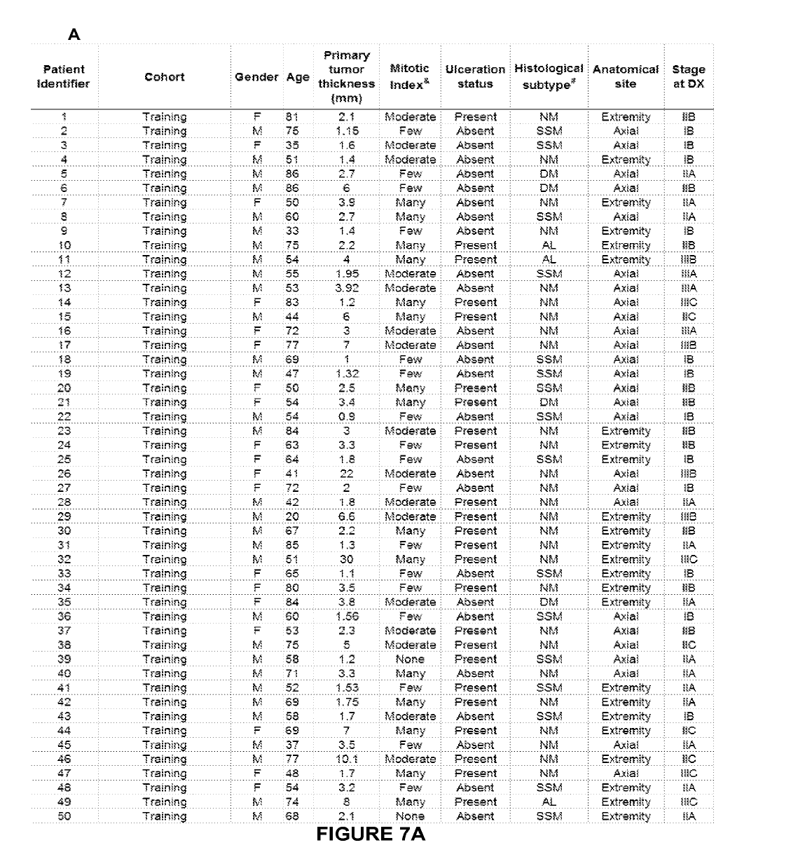

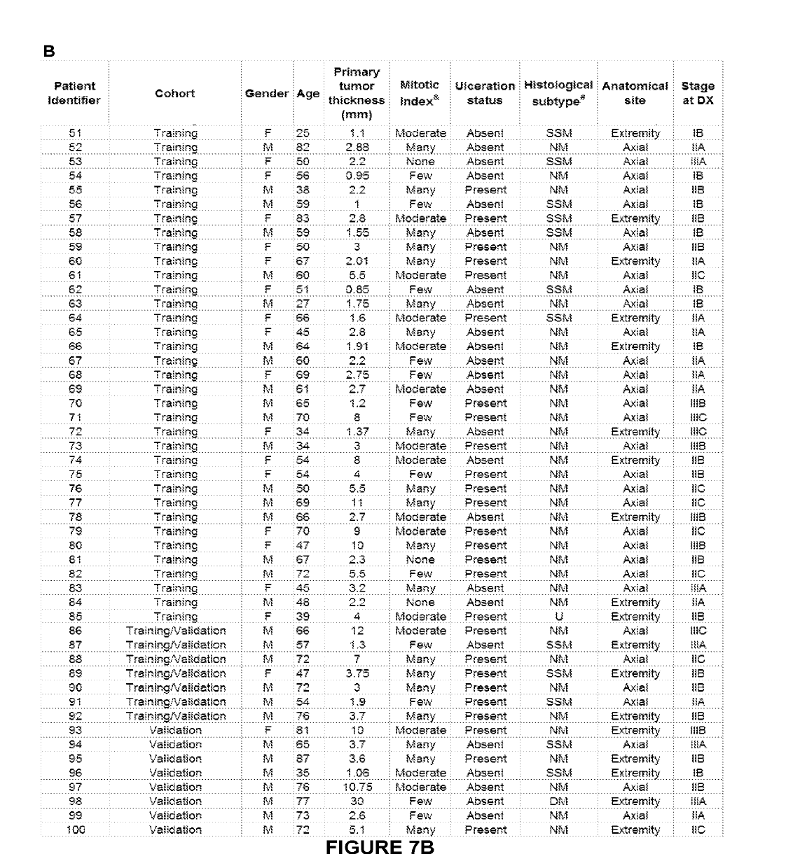

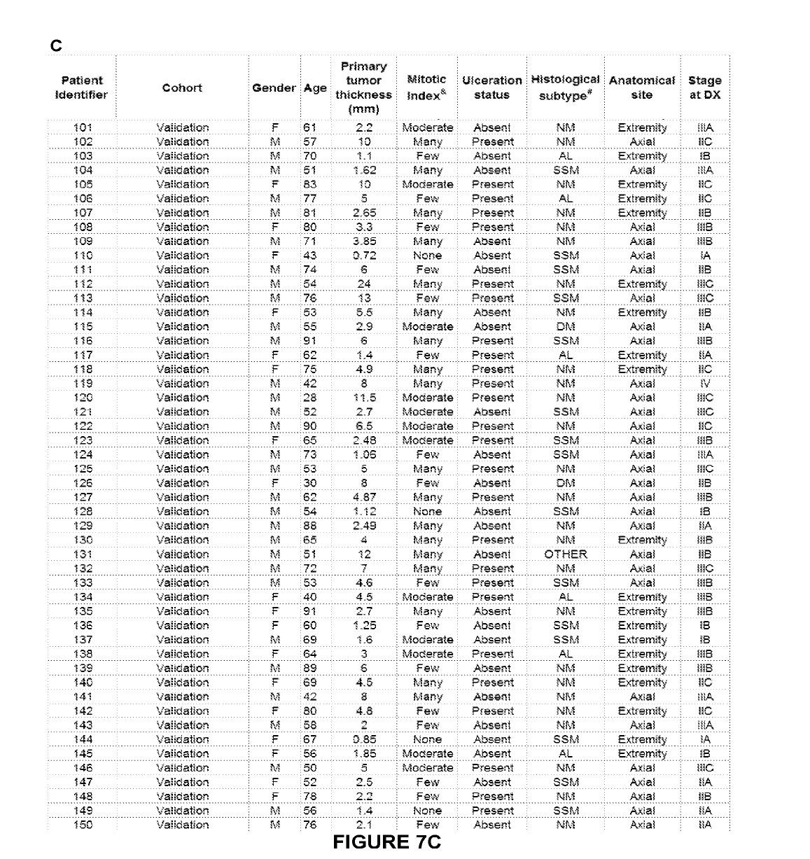

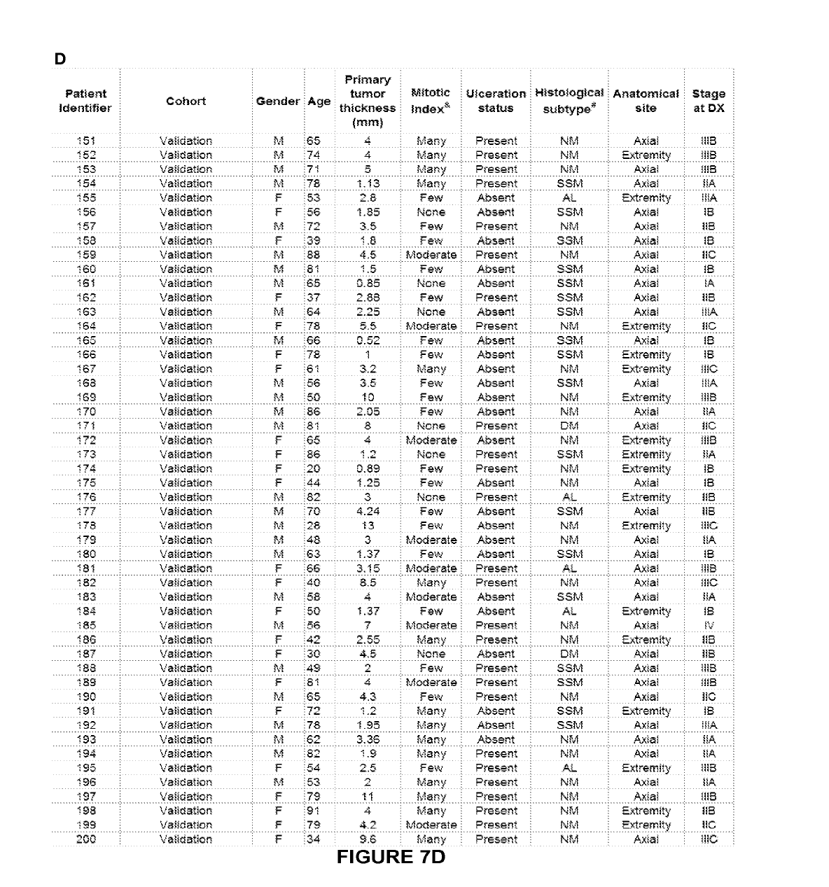

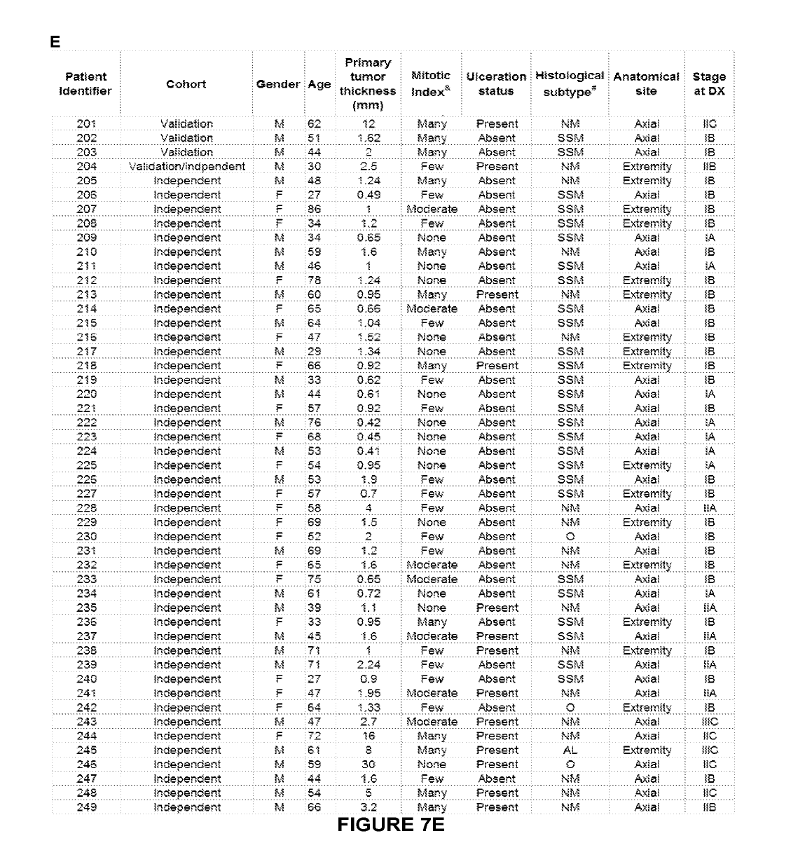

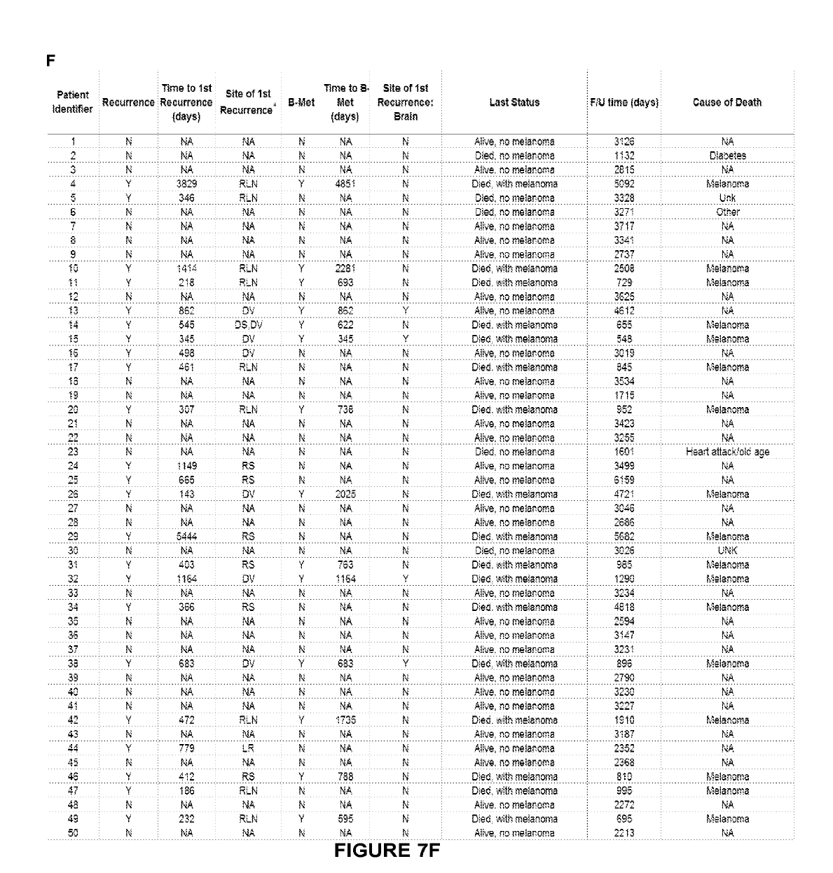

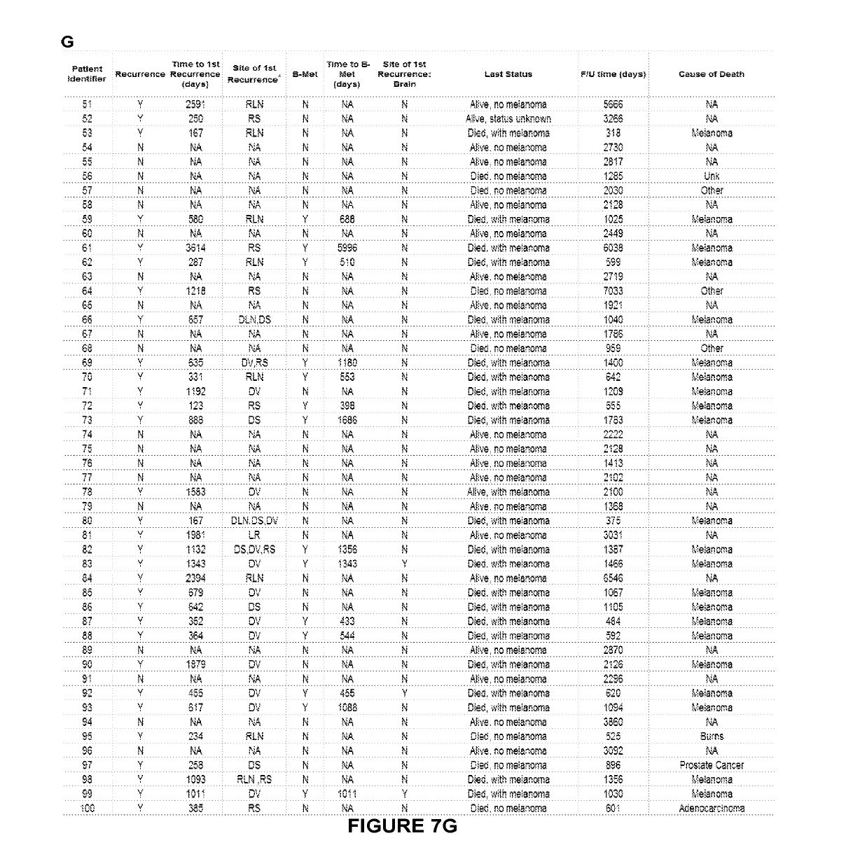

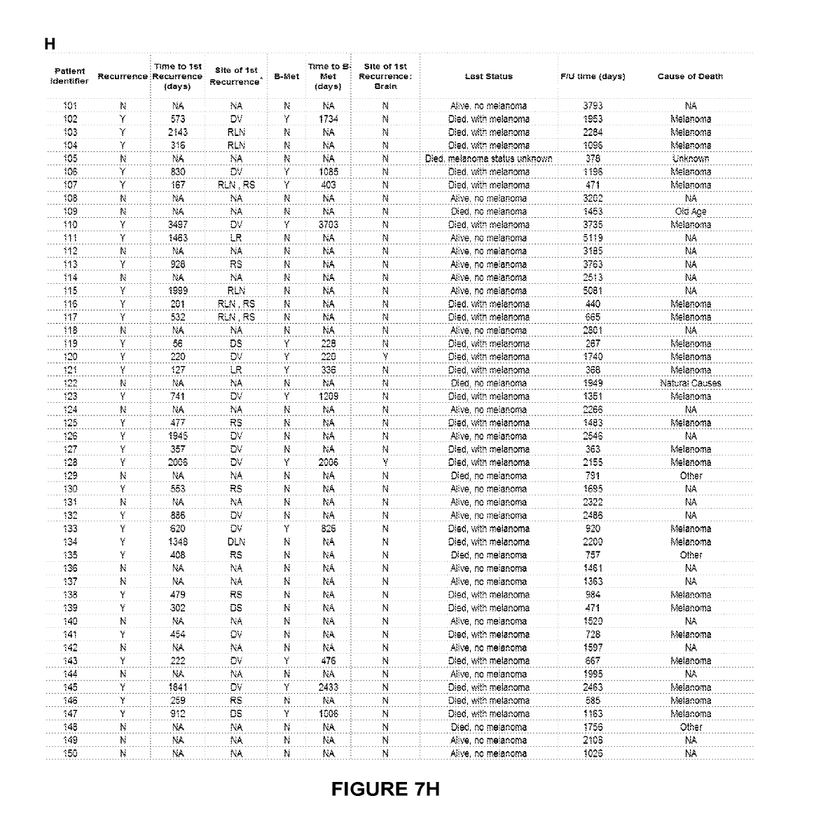

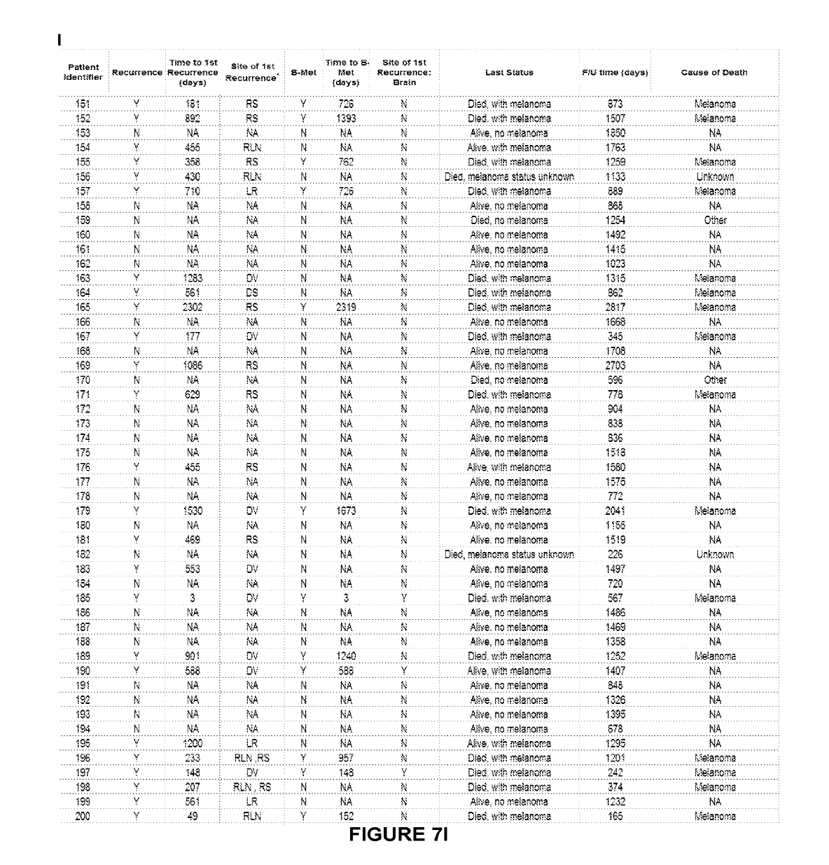

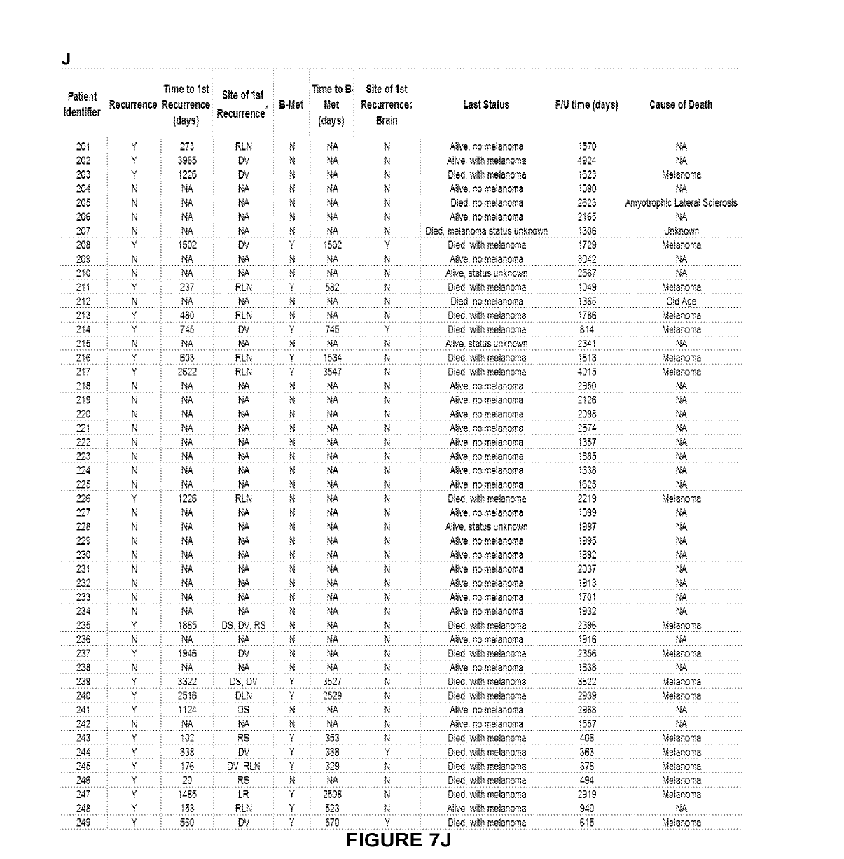

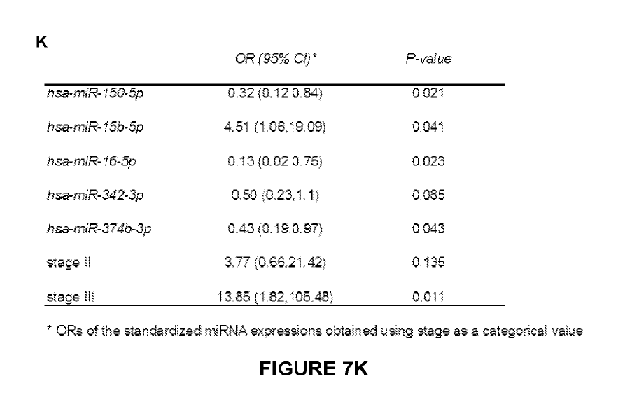

Development of a technical platform amenable to reproducible and precise quantification (such as RT-qPCR) and performance in a CLIA-certified laboratory would be beneficial steps in such stratification. This present invention further discloses a miRNA quantification platform amenable to a clinical assessment, useful as a prognostic test in the clinical setting. In support of this translation, there is a good correlation between risk scores for identical samples derived from microarray and RT-qPCR data (r=0.412, p<0.001, FIG. 6).

This further supports the robustness of the present invention and demonstrates feasibility this and other embodiments of miRNA quantification platforms. The addition of a prognostic assay based on the described miRNA expression signature to currently used staging criteria has the potential to improve accuracy of primary melanoma patient prognoses and aid their clinical management, including selection for adjuvant treatment or clinical trials of adjuvant therapies.

In summary, the prognostic value of miRNA expression in primary melanoma tissues was analyzed from three patient cohorts. A prognostic miRNA classifier that robustly and accurately stratifies early stage primary melanoma patients by their risk of developing B-Met is disclosed, improving upon the discriminatory accuracy of existing clinical variable based prediction models. The present invention represents the first useful molecular B-Met prognostic assay for melanoma and, as such, will improve clinical care and outcomes of primary melanoma patients.

LIST OF SEQUENCES:

| | sequence for mature | |
|---|---|---|
| | human miRNA | SEQ |
| NAME | (miRBase Accession No.) | ID NO: |
| hsa-miR-150-5p | UCUCCCAACCCUUGUACCAGUG | 1 |
| hsa-miR-15b-5p | UAGCAGCACAUCAUGGUUUACA | 2 |
| hsa-miR-16-5p | UAGCAGCACGUAAAUAUUGGCG | 3 |
| hsa-miR-374b-3p | CUUAGCAGGUUGUAUUAUCAUU | 4 |

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

All patents, applications, publications, test methods, literature, and other materials cited herein are hereby incorporated by reference in their entirety as if physically present in this specification.

1. A method for predicting the likelihood of developing melanoma brain metastasis (B-Met) in a subject diagnosed with primary melanoma, said method comprising:
a. determining the levels of miR-150-5p, miR-15b-5p, miR-16-5p, and miR-374b-3p in a primary melanoma sample collected from the subject;
b. calculating a B-Met prognostic score based on a weighted summation of the miRNA levels determined in step (a) and melanoma stage of the primary melanoma sample collected from the subject;
c. comparing the B-Met prognostic score calculated in step (b) with a corresponding control value;
d. (i) identifying the subject as being at high risk of developing B-Met if the B-Met prognostic score calculated in step (b) is higher than the corresponding control value or (ii) identifying the subject as being at low risk of developing B-Met if the B-Met prognostic score calculated in step (b) is same or lower than the corresponding control value.

2. A method for predicting the likelihood of developing melanoma brain metastasis (B-Met) in a subject diagnosed with primary melanoma, said method comprising:
a. determining the levels of miR-150-5p, miR-15b-5p, miR-16-5p, and miR-374b-3p in a primary melanoma sample collected from the subject by an RT-PCR-based assay;
b. calculating a B-Met prognostic score based on a weighted summation of the miRNA levels determined in step (a) and melanoma stage of the primary melanoma sample collected from the subject using the formula
Score=−0.51 miR-150+0.65 miR15b−0.96miR-16−0.24 miR-374+0.47*stage;
c. comparing the B-Met prognostic score calculated in step (b) with a corresponding control value;
d. (i) identifying the subject as being at high risk of developing B-Met if the B-Met prognostic score calculated in step (b) is higher than the corresponding control value or (ii) identifying the subject as being at low risk of developing B-Met if the B-Met prognostic score calculated in step (b) is same or lower than the corresponding control value.

3. A method for predicting the likelihood of developing melanoma brain metastasis (B-Met) in a subject diagnosed with primary melanoma, said method comprising:
a. determining the levels of miR-150-5p, miR-15b-5p, miR-16-5p, and miR-374b-3p in a primary melanoma sample collected from the subject by a microarray-based assay;
b. calculating a B-Met prognostic score based on a weighted summation of the miRNA levels determined in step (a) and melanoma stage of the primary melanoma sample collected from the subject using the formula
Score=−0.51 miR-150+0.65 miR15b−0.96 miR-16−0.24 miR-374+0.47*stage;
c. comparing the B-Met prognostic score calculated in step (b) with a corresponding control value;
d. (i) identifying the subject as being at high risk of developing B-Met if the B-Met prognostic score calculated in step (b) is higher than the corresponding control value or (ii) identifying the subject as being at low risk of developing B-Met if the B-Met prognostic score calculated in step (b) is same or lower than the corresponding control value.

4. The method of any one of claims 1-3, wherein the subject has been diagnosed with primary cutaneous melanoma.

5. The method of any one of claims 1-3, wherein the subject has been diagnosed with stage I, stage II or stage III primary melanoma.

6. The method of any one of claims 1-3, wherein the melanoma stage is determined according to the American Joint Committee on Cancer (AJCC) 2009 final guidelines.

7. The method of any one of claims 1-3, further comprising determining melanoma stage of the primary melanoma sample collected from the subject prior to step (b).

8. The method of claim 7, wherein the melanoma stage is determined according to the American Joint Committee on Cancer (AJCC) 2009 final guidelines.

9. The method of any one of claims 1-3, wherein the control value is a predetermined standard.

10. The method of any one of claims 1-3, wherein the control value is the mean of prognostic probabilities calculated for the same miRNA in several similarly prepared melanoma samples isolated from subjects with low risk of developing B-Met.

11. The method of any one of claims 1-3, further comprising normalizing the levels of miRNA determined in step (a) to one or more normalizer RNA.

12. The method of claim 11, wherein the normalizer RNA is miRNA.

13. The method of claim 11, further comprising
(i) calculating the mean expression Ct/Cp for the one or more normalizer miRNA to generate a normalizer Ct/Cp;
(ii) subtracting test miRNA Ct/Cp from the normalizer Ct/Cp calculated in step (i) to generate normalized delta Ct/Cp, and
(iii) using the normalized delta Ct/Cp calculated in step (ii) in step (b).

14. The method of claim 11, wherein the combination of let-7e-5p, miR-30d-5p, and miR-423-3p is used as the normalizer.

15. The method of any one of claims 1-3, further comprising recruiting the subject in a clinical trial.

16. The method of any one of claims 1-3, further comprising conducting a regular surveillance of the subject determined in step (e) as being at high risk of developing B-Met for the presence of B-Met.

17. The method of claim 16, wherein the surveillance includes brain imaging and/or clinical evaluation.

18. The method of any one of claims 1-3, further comprising increasing the frequency of surveillance of the subject determined in step (e) as being at high risk of developing B-Met, as compared to the subject determined as being at low risk of developing B-Met.

19. The method of claim 18, wherein the surveillance includes brain imaging and/or clinical evaluation.

20. The method of any one of claims 1-3, further comprising conducting an extensive primary tumor staging of the subject determined in step (e) as being at high risk of developing B-Met.

21. The method of any one of claims 1-3, further comprising administering to the subject determined in step (e) as being at high risk of developing B-Met a treatment specifically targeted at prevention or treatment of B-Met.

22. The method of claim 21, wherein the treatment comprises administering to the subject one or more agents selected from the group consisting of Interferon alpha, a BRAF inhibitor, a MEK inhibitor, imatinib, nilotinib, dacarbazine, temozolomide, and an immunotherapy agent.

23. The method of claim 22, wherein the BRAF inhibitor is vemurafenib or dabrafenib.

24. The method of claim 22, wherein the MEK inhibitor is trametinib.

25. The method of claim 22, wherein the immunotherapy agent is selected from the group consisting of anti-CTLA4, anti-PD1, and anti-PD-Ll.

26. The method of claim 21, wherein the treatment is a cranial irradiation.

27. The method of any one of claims 1-3, which method further comprises a step of collecting the primary melanoma sample from the subject prior to step (a).

28. The method of claim 1, wherein the levels of the miRNA are determined using a method selected from the group consisting of hybridization, array-based assays, RT-PCR-based assays, and sequencing.

29. The method of any one of claims 1-3, wherein, prior to determining miRNA level, the miRNA is purified from the melanoma sample.

30. The method of any one of claims 1-3, further comprising the step of reducing or eliminating degradation of the miRNA.

31. A method for prevention or treatment of melanoma brain metastasis (B-Met) in a subject in need thereof comprising increasing the level and/or activity of one or more miRNA selected from the group consisting of miR-150-5p, miR-16-5p, and miR-374b-3p, in the melanoma cells of the subject.

32. The method of claim 31, wherein increasing the level and/or activity of said one or more miRNA is achieved by a method selected from the group consisting of over-expressing miRNA or mature miRNA mimic, sense-based oligonucleotides, and modified-oligonucleotide mimics.

33. A method for prevention or treatment of melanoma brain metastasis (B-Met) in a subject in need thereof comprising decreasing the level and/or activity of miR-15b-5p in the melanoma cells of the subject.

34. The method of claim 33, wherein decreasing the level and/or activity of miR-15b-5p is achieved using an RNAi molecule or an antisense oligonucleotide.

35. A method for quantifying tumor-infiltrating lymphocytes (TILs) in a tumor sample collected from a subject, wherein the tumor expresses low levels or does not express miR-150-5p, said method comprising determining the level of miR-150-5p in the tumor sample.

36. A method for quantifying lymphocyte response to a tumor in a subject, wherein the tumor expresses low levels or does not express miR-150-5p, said method comprising determining the level of miR-150-5p in a tumor sample collected from the subject.

37. The method of claim 35 or claim 36, wherein the level of miR-150-5p in the tumor sample is normalized to the level of miR-150-5p in the surrounding stroma.

38. The method of claim 35 or claim 36, wherein the tumor is melanoma.

39. The method of claim 35 or claim 36, wherein the level of miR-150-5p is determined using a method selected from the group consisting of hybridization, array-based assays, RT-PCR-based assays, and sequencing.

40. The method of claim 35 or claim 36, wherein, prior to determining the level of miR-150-5p, miRNA is purified from the tumor sample.

41. The method of claim 35 or claim 36, further comprising the step of reducing or eliminating degradation of miRNA in the tumor sample.

42. The method of any one of claims 1-3, 31, 33, 35, and 36, wherein the subject is human.

43. The method of any one of claims 1-3, 31, 33, 35, and 36, wherein the subject is an experimental animal.

44. A kit comprising primers and/or probes specific for miR-150-5p, miR-15b-5p, miR-16-5p, and miR-374b-3p.

45. The kit of claim 44, further comprising miRNA isolation or purification means.

46. The kit of claim 44, further comprising instructions for use.

47. The method of claim 31 or 33, further comprising administering to the subject an additional treatment specifically targeted at prevention or treatment of B-Met.

48. The method of claim 47, wherein the additional treatment comprises administering to the subject one or more agents selected from the group consisting of Interferon alpha, a BRAF inhibitor, a MEK inhibitor, imatinib, nilotinib, dacarbazine, temozolomide, and an immunotherapy agent.

49. The method of claim 48, wherein the BRAF inhibitor is vemurafenib or dabrafenib.

50. The method of claim 48, wherein the MEK inhibitor is trametinib.

51. The method of claim 48, wherein the immunotherapy agent is selected from the group consisting of anti-CTLA4, anti-PD1, and anti-PD-Ll.

52. The method of claim 47, wherein the treatment is a cranial irradiation.
